(19) United States
(12) Patent Application Publication
(10) Pub. No.: US 2006/0271032 A1
(43) Pub. Date: Nov. 30, 2006

(54) Ablation instruments and methods for performing abalation

(76) Inventors: Albert K. Chin, Palo Alto, CA (US); Peter Callas, Redwood City, CA (US); Shuji Uemura, San Francisco, CA (US); Geoffrey H. Willis, Redwood City, CA (US)

Correspondence Address: LAW OFFICE OF ALAN W. CANNON, 834 SOUTH WOLFE ROAD, SUNNYVALE, CA, 94086, US

(21) Appl. No.: 11/137,987

(22) Filed: May 26, 2005

(51) Int. Cl. A61B 18/14

(52) U.S. Cl. 606/41

(57) ABSTRACT

Methods, devices and instruments provided for performing ablation transmurally across the wall of an organ. Devices may directly access tissue to be ablated through direct access openings formed in the patient and, optionally, an organ where the ablation is to be performed. Instruments facilitating making openings, dissecting, and delivery of ablation instruments are also described.

### FIELD OF THE INVENTION

The present invention relates to the field of surgical devices, and more particularly to ablation devices and methods.

### BACKGROUND OF THE INVENTION

Various medical conditions, diseases and dysfunctions may be treated by ablation, using various ablation devices and techniques. Ablation is generally carried out to kill or destroy tissue at the site of treatment to bring about an improvement in the medical condition being treated.

In the cardiac field, cardiac arrhythmias, and particularly atrial fibrillation are conditions that have been treated with some success by various procedures using many different types of ablation technologies. Atrial fibrillation continues to be one of the most persistent and common of the cardiac arrhythmias, and may further be associated with other cardiovascular conditions such as stroke, congestive heart failure, cardiac arrest, and/or hypertensive cardiovascular disease, among others. Left untreated, serious consequences may result from atrial fibrillation, whether or not associated with the other conditions mentioned, including reduced cardiac output and other hemodynamic consequences due to a loss of coordination and synchronicity of the beating of the atria and the ventricles, possible irregular ventricular rhythm, atrioventricular valve regurgitation, and increased risk of thromboembolism and stroke.

As mentioned, various procedures and technologies have been applied to the treatment of atrial arrhythmias/fibrillation. Drug treatment is often the first approach to treatment, where it is attempted to maintain normal sinus rhythm and/or decrease ventricular rhythm. However, drug treatment is often not sufficiently effective and further measures must be taken to control the arrhythmia.

Electrical cardioversion and sometimes chemical cardioversion have been used, with less than satisfactory results, particularly with regard to restoring normal cardiac rhythms and the normal hemodynamics associated with such.

A surgical procedure known as the MAZE III (which evolved from the original MAZE procedure) procedure involves electrophysiological mapping of the atria to identifying macroreentrant circuits, and then breaking up the identified circuits (thought to be the drivers of the fibrillation) by surgically cutting or burning a maze pattern in the atrium to prevent the reentrant circuits from being able to conduct therethrough. The prevention of the reentrant circuits allows sinus impulses to activate the atrial myocardium without interference by reentering conduction circuits, thereby preventing fibrillation. This procedure has been shown to be effective, but generally requires the use of cardiopulmonary bypass, and is a highly invasive procedure associated with high morbidity.

Other procedures have been developed to perform transmural ablation of the heart wall or adjacent tissue walls. Transmural ablation may be grouped into two main categories of procedures: endocardial and epicardial. Endocardial procedures are performed from inside the wall (typically the myocardium) that is to be ablated, and is generally carried out by delivering one or more ablation devices into the chambers of the heart by catheter delivery, typically through the arteries and/or veins of the patient. Epicardial procedures are performed from the outside wall (typically the myocardium) of the tissue that is to be ablated, often using devices that are introduced through the chest and between the pericardium and the tissue to be ablated. However, mapping may still be required to determine where to apply an epicardial device, which may be accomplished using one or more instruments endocardially, or epicardial mapping may be performed. Various types of ablation devices are provided for both endocardial and epicardial procedures, including radiofrequency (RF), microwave, ultrasound, heated fluids, cryogenics and laser. Epicardial ablation techniques provide the distinct advantage that they may be performed on the beating heart without the use of cardiopulmonary bypass.

When performing procedures to treat atrial fibrillation, an important aspect of the procedure generally is to isolate the pulmonary veins from the surrounding myocardium. The pulmonary veins connect the lungs to the left atrium of the heart, and join the left atrial wall on the posterior side of the heart. This location creates significant difficulties for endocardial ablation devices delivered endovascularly, e.g., ablation catheter systems. Although many of the other lesions can be created from within the right atrium, the pulmonary venous lesions must be created in the left atrium, requiring either a separate arterial access point or a transeptal puncture from the right atrium. Ablation catheter systems require, by definition, flexible, elongated delivery catheters that may be difficult to manipulate into the complex geometries required for forming the pulmonary venous lesions and to maintain in those positions against the wall of a beating heart during lesion formation. This is very time-consuming and can result in lesions which do not completely encircle the pulmonary veins or which contain gaps and discontinuities. Furthermore, the complication of pulmonary vein stenosis may occur if the ablation catheter ablates the pulmonary vein or a portion thereof, rather than ablation only atrial tissue surrounding the pulmonary vein. Visualization of endocardial anatomy and endovascular devices, using ablation catheter systems, may not be sufficient to accurately determine the precise position(s) of the ablation device(s) for accurate placement of lesions.

Thus, there is a continuing need for devices, techniques, systems and procedures for forming lesions in accurate, intended locations, that are sufficiently transmural and continuous to effectively prevent reentrant signals, as well as foci-originated signals, including those emanating from the pulmonary veins.

### SUMMARY OF THE INVENTION

Methods and device are provided for performing ablation transmurally across the wall of an organ, including preparing an opening in a patient to provide direct access to the wall of the organ; preparing an opening through the organ; inserting an ablation device through the opening in the patient and the opening through the organ; approximating a target area of an inner wall of the organ with an ablation element of the ablation device; and ablating the target area to create a lesion.

Methods and devices are provided for performing atrial ablation by making an opening in a patient to provide direct access to the heart of the patient; making an opening in the pericardium; inserting an ablation device through the opening in the patient and the opening in the pericardium; approximating a target area of a wall of the organ with an ablation element of the ablation device; and ablating the target area to create a lesion.

Further, methods and devices for performing ablation are provided to include steps of inserting an ablation device comprising a rigid or malleable tube and at least one ablation element at a distal end portion thereof through an opening in the chest of a patient and through the atrium; viewing the location and placement of the distal end of the ablation device through an endoscope passing axially therethrough; and ablating tissue at a target location on the endocardium in the atrium.

Ablation devices are provided for directly accessing a surgical site to perform ablation on a targeted tissue, wherein such a device includes an elongated rigid or malleable tube having a distal end portion and a proximal end portion, said elongated tube having sufficient length so that at least a proximal end of the proximal end portion extends out of a patient when a distal end of the distal end portion contacts the targeted tissue; an endoscope axially received within said elongated rigid or malleable tube; a transparent tip closing the distal end of said distal end portion, wherein said transparent tip enables direct viewing through the distal end of the device using said endoscope; and at least one ablation element mounted on said device at said distal end portion.

Embodiments of the invention include an ablation device for directly accessing a surgical site to perform ablation on a targeted tissue, including an elongated rigid or malleable tube having a distal end portion and a proximal end portion, said elongated tube having sufficient length so that at least a proximal end of the proximal end portion extends out of a patient when a distal end of the distal end portion contacts the targeted tissue; and a variable diameter tip mounted to said distal end portion of said tube, said variable diameter tip adapted to contact tissue and apply at least one of an energy or chemical to the tissue to perform ablation of the tissue.

Still further, an ablation device for directly accessing a surgical site to perform ablation on a targeted tissue is provided, including an elongated rigid or malleable tube; a transparent distal tip mounted at a distal end of said tube; a balloon member axially mounted over a distal end portion of said tube, proximal of said distal tip and fluidly connected to an opening through said tube for inflation of said balloon member by delivering pressurized fluid through said tube; and an ablation element located within said balloon member.

A device for facilitating the formation of an opening through an organ and for facilitating the delivery of at least one instrument through the opening is provided to include an elongated main tube having proximal and distal ends; a sewing ring located about said distal end; and a one-way valve located within a proximal end portion of said main tube.

A dissection instrument is provided to include an elongated rigid or malleable tube having a distal end portion and a proximal end portion, said elongated tube having sufficient length so that at least a proximal end of the proximal end portion extends out of a patient when a distal end of the distal end portion contacts tissue as it is dissected; an endoscope axially received within said elongated rigid or malleable tube; a transparent blunt tip closing the distal end of said distal end portion, wherein said transparent blunt tip enables direct viewing through the distal end of the device using said endoscope; and a transparent member having a sharp configuration mounted between said blunt tip and a distal end of said endoscope.

An ablation device for directly accessing a surgical site to perform ablation on a targeted tissue, as disclosed, includes: an elongated rigid or malleable tube having a distal end portion and a proximal end portion, said elongated tube having sufficient length so that at least a proximal end of the proximal end portion extends out of a patient when a distal end of the distal end portion contacts the targeted tissue; an endoscope axially received within said elongated rigid or malleable tube; a transparent tip closing the distal end of said distal end portion, wherein said transparent tip enables direct viewing through the distal end of the device using said endoscope; and at least one ablation element mounted on a distal end portion of said device.

These and other advantages and features of the invention will become apparent to those persons skilled in the art upon reading the details of the methods, devices and instruments as more fully described below.

### BRIEF DESCRIPTION OF THE DRAWINGS

FIG. 1A illustrates a purse-string suture placed around a section of the free border of an atrial appendage.

FIG. 1B shows an enlarged, detailed portion of FIG. 1A showing the formation of the purse-string suture being formed in the atrial appendage, in more detail.

FIG. 2 illustrates a cutaway view of an ablation instrument having been inserted through an atrial appendage, and then manipulated/maneuvered to cannulate a pulmonary vein ostium.

FIG. 3A shows a perspective view and FIG. 3B shows an end view of an example of an ablation instrument according to the present invention.

FIG. 3C illustrates a lesion formed about an ostium and circumferentially spaced from the ostium.

FIG. 4A show a perspective view of another example of an ablation instrument that includes an elongated tube or shaft that houses or surrounds an endoscope.

FIG. 4B. shows a partial perspective view of the device shown in FIG. 4A, wherein a sliding ring is in a retracted position so that tip/lens of the device protrudes distally therefrom

FIG. 4C is a view similar to FIG. 4B, except that the sliding ring is slid distally with respect to its position shown in FIG. 4B.

FIG. 4D is another partial view of the device of FIGS. 4A-4C illustrating (in phantom lines) at least one conduit provided within the sliding ring and extending proximally out of the device, through which positive pressure irrigation (e.g., such as by saline) may be applied to the working space.

FIGS. 4E-4F show partial views of a device similar to that of FIGS. 4A-4D, where the sliding ring is expandable.

FIGS. 5A and 5B are partial views of another example of an ablation instrument having a working end portion that is adjustable in size.

FIG. 5C illustrates a variation of the device of FIGS. 5A-5B that includes a protrusion on a distal surface of the expandable member.

FIGS. 6A-6B illustrate how a view through an instrument can be obscured by blood if the tip/lens of the instrument is too small relative to an ostium that it is attempting to cannulate.

FIG. 6C illustrates the tendency of a tip/lens of an ablation instrument used to visualize a pulmonary vein ostium to flatten out against the atrial wall if it lacks sufficient rigidity.

FIG. 6D illustrates the view observed through the endoscope of the instrument when the tip flattens out as shown in FIG. 6C.

FIG. 6E illustrates a device having a tip/lens that is properly sized and has sufficient rigidity to approximate/cannulate an ostium in the desired manner.

FIG. 6F illustrates a view obtained through the instrument of FIG. 6E when correctly positioned as in FIG. 6E.

FIG. 6G illustrates an example of an ablation instrument having an expandable distal tip, showing both the deflated state of the distal tip and an inflated configuration (in phantom).

FIG. 6H is a plan drawing of an device having an expandable distal tip.

FIG. 6I is a partial sectional view taken along line 6I-6I in FIG. 6H.

FIG. 6J is a partial perspective view of another ablation device according to the present invention, shown in a contracted configuration.

FIG. 6K is a partial perspective view of the ablation device of FIG. 6J, shown in an expanded configuration.

FIG. 6L illustrates an example of an expandable frame that may be employed in the device of FIGS. 6K-6K.

FIG. 6M illustrates an end view of the frame of FIG. 6L.

FIG. 6N is a partial view illustrating cinching down of the expanded frame shown in FIG. 6L, when tension is applied through the pull wire.

FIG. 7A illustrates the principle that energy applied to a surface of a tissue wall will travel depthwise (i.e., through the thickness of the wall) to approximately the same distance y as the distance x that the energy travels radially outward (i.e., along the tissue surface) from the point of application.

FIGS. 7B and 7C are illustrations showing an end view and a side view, respectively, of a monitoring element mounted with respect to an ablation element at a radial distance that approximately equal to the thickness of the wall of the tissue to be ablated.

FIG. 8A illustrates another example of an ablation device/instrument having a variable diameter tip.

FIGS. 8B and 8C illustrate this principle for expanding the distal tip of the device of FIG. 8A, where FIG. 8B shows the most expanded configuration, and FIG. 8C shows the edges having been rotated, relative to one another in the directions of the arrows shown, which results in a reduction of the outside diameter of the tip of the device.

FIG. 8D is a partial perspective view of the ablation instrument of FIG. 8A showing the distal tip in a larger diameter configuration than that shown in FIG. 8E, where the configuration in FIG. 8D corresponds to what was described with regard to FIG. 8B and the configuration shown in FIG. 8E corresponds to what was described with regard to FIG. 8C.

FIG. 8E illustrates the connection of an expandable coil to tubes of the device of FIG. 8A.

FIG. 8F is a partial perspective view showing the connection of the expandable coil to one of the tubes of the device of FIG. 8A.

FIG. 8G shows reinforcement of the connection between the coil and the tube of the device of FIG. 8A, using shrink tubing.

FIGS. 8H and 8I illustrate an end view of tubes of the device of FIG. 8A, with the coil attached thereto, and showing the attachments of the coil to the tubes, wherein FIG. 8H shows an enlarged diameter configuration and FIG. 8I shows a reduced diameter configuration.

FIG. 8J illustrates a sheet of material used to make the tip of the device of FIG. 8A, shown in planar form.

FIG. 8K shows the sheet material of FIG. 8J attached to a coil to form the tip of the device of FIG. 8A.

FIG. 8L illustrates a sealing sleeve provided over the distal end portion of the outer tubing of the device of FIG. 8A, that attaches the proximal end of the tip to prevent blood/fluid flow into the coil and inside of instrument/device.

FIG. 8M is a partial view illustrating one example of a control grip in an unlocked position or configuration.

FIG. 8N is a partial view illustrating the example of FIG. 8M in a locked position or configuration.

FIG. 8O is a partial view illustrating another examples of a control grip.

FIG. 9A is a partial view illustrating another example of an ablation instrument having a variable diameter tip.

FIG. 9B illustrates an example of preformed curved control member that may be used to drive the expansion of the expandable tip of the device shown in FIG. 9A.

FIG. 9C illustrates distal movement of control members (in the direction of arrow 59) relative to the tube of the device and the resulting expansion of the tip member.

FIG. 9D is a partial view illustrating an endoscope axially positioned within the device shown in FIG. 9C.

FIG. 9E is a partial view illustrating separate tubing provided between the endoscope and tube of the device in FIG. 9D, to guide the movements of the control members.

FIG. 10A is a partial perspective view illustrating another ablation instrument with a varying diameter tip portion.

FIG. 10B is a partial view illustrating the device of FIG. 10A in an expanded configuration.

FIG. 10C is a partial view showing a device as in FIGS. 10A and 10B, wherein an endoscope mounted therein is axially translatable with respect to the outer tubing and tip of the device.

FIG. 10D is a partial view of a device similar to that shown in FIG. 10C, wherein the endoscope is additionally flexible or articulating to allow panning of the view.

FIG. 10E is a partial view illustrating an ablation instrument 10 of the type described in FIGS. 10A-10D, in position to perform an ablation.

FIG. 11A is a perspective view of an ablation instrument configured for applying ultrasonic energy to perform ablation.

FIG. 11B is a partial view illustrating an expandable member of the device of FIG. 11A in a deflated or contracted state.

FIG. 11C is a partial view illustrating an expandable member of the device of FIG. 11A in an inflated or expanded state.

FIG. 11D is an isolated, perspective view of a balloon mount segment that may be included in the instruments shown in FIGS. 11A-11C.

FIG. 12A is a partial view illustrating another example of an ablation instrument having a tip portion that is adjustable in size.

FIG. 12B shows an end view of the instrument of FIG. 12A in a smallest diameter configuration.

FIG. 12C is a partial view illustrating the instrument of FIG. 12A, wherein the tip has been expanded relative to that shown in FIG. 12A.

FIGS. 12E and 12F illustrate contracted and expanded end views, respectively, of an instrument similar to that shown in FIGS. 12A-12D, that includes a coiled ring as an alternative to the split ring portions of the instrument of FIGS. 12A-12D.

FIG. 12G and inner an outer tube arrangement configured to house an endoscope and to provide a lumen through which an expandable member is inflated or expanded.

FIG. 12H shows the tubing arrangement of FIG. 12G in separated form.

FIG. 12I illustrates a partial perspective view of an ablation instrument of a type described above with regard to FIGS. 12A-12H, within which an endoscope is provided.

FIG. 12J shows a perspective view of the device of FIG. 12I, including a camera and a pressurized fluid source.

FIG. 12K shows the instrument of FIGS. 12I-12J, wherein the expandable member has been slightly deflated and the endoscope, together with the expandable member, were retracted slightly with respect to the outer tube of the instrument.

FIGS. 12L and 12M show a partial perspective view and a partial side view, respectively, of a device showing fixation of ring to split tubing via a rivet or similar mechanical fixation.

FIGS. 12N and 12O show a partial perspective view and a partial side view, respectively, of a device showing fixation of ring to split tubing via a suture.

FIG. 12P shows a partial perspective view of a device showing fixation of ring to split tubing via a tab and slot arrangement.

FIGS. 13A and 13B show a perspective illustration and an end view illustration, respectively of ring 86, as shown in FIG. 12K.

FIG. 13A is a perspective illustration and FIG. 13B is an end view illustration of another example of an ablation instrument having a single ablation element.

FIG. 14A is a perspective illustration, and FIG. 14B is a distal end illustration of an ablation instrument configured to drag the tip portion in order to form a lesion via ablation.

FIG. 14C is a partial view illustrating the wiring and contacts of the ablation element of the instrument shown in FIGS. 14A-14B.

FIG. 15A is a partial view of a telescoping ablation instrument shown with the ablation element “telescoped out”.

FIG. 15B is a partial view of the instrument shown in FIG. 15A, shown with the ablation element “telescoped in”.

FIG. 15C is a partial view of another example of a telescoping ablation instrument shown with the ablation element “telescoped out”.

FIG. 15D is a partial view of the instrument shown in FIG. 15C, shown with the ablation element “telescoped in”.

FIG. 16 shows a device for facilitating the delivery of an instrument through an opening leading to a surgical site.

FIG. 17 shows a tubular cutter to be inserted through the device shown in FIG. 16, to cut an opening through the tissue that the device is attached to.

FIG. 18A is a partial view illustrating an endoscope positioned so that the distal end of the endoscope is pulled back or retracted form the radial confines of the tip of the ablation instrument shown.

FIG. 18B illustrates a bright ring visual artifact that may occur when viewing through an endoscope with an arrangement as shown in FIG. 18A.

FIG. 18C is a partial view illustrating an ablation instrument similar to that show in FIG. 18A and additionally having a tapered or conical tip provided within the blunt or hemispherical tip.

FIG. 19 is a partial view illustrating a dissection instrument including a rigid, transparent, blunt tip that enables viewing of the progress of the dissection procedure through an endoscope, and a tapered or conical tip provided within the blunt tip.

FIG. 20A is a partial sectional view illustrating another example of a dissection instrument, including an aspiration/irrigation channel to extend through the tip portion of the instrument.

FIG. 20B is a partial sectional view of the dissection instrument shown in FIG. 20A, with further illustration of a stylet having been slid through the aspiration/irrigation channel.

### DETAILED DESCRIPTION OF THE INVENTION

Before the present devices, methods and systems are described, it is to be understood that this invention is not limited to particular devices and method steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a lesion” includes a plurality of such lesions and reference to “the electrode” includes reference to one or more electrodes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The following devices described are for performing ablation, particularly for endocardial ablation techniques, although they may also be used for ablation in other tissue or organs of an organism, as well as for epicardial applications. More particularly, these devices are configured to perform endocardial ablation in a more direct, less invasive manner than what is currently practiced. Although not limited thereto, a particularly beneficial technique according to the present invention is the performance of atrial ablation on the beating heart under closed chest conditions. The devices may be alternatively used to perform atrial ablation on a stopped heart under closed chest conditions, or upon a stopped or beating heart under open chest conditions. Of course, ablation of other tissue, such as in the ventricles, or other tissues may be practiced. Still further, the present devices may be used to practice epicardial ablation procedures.

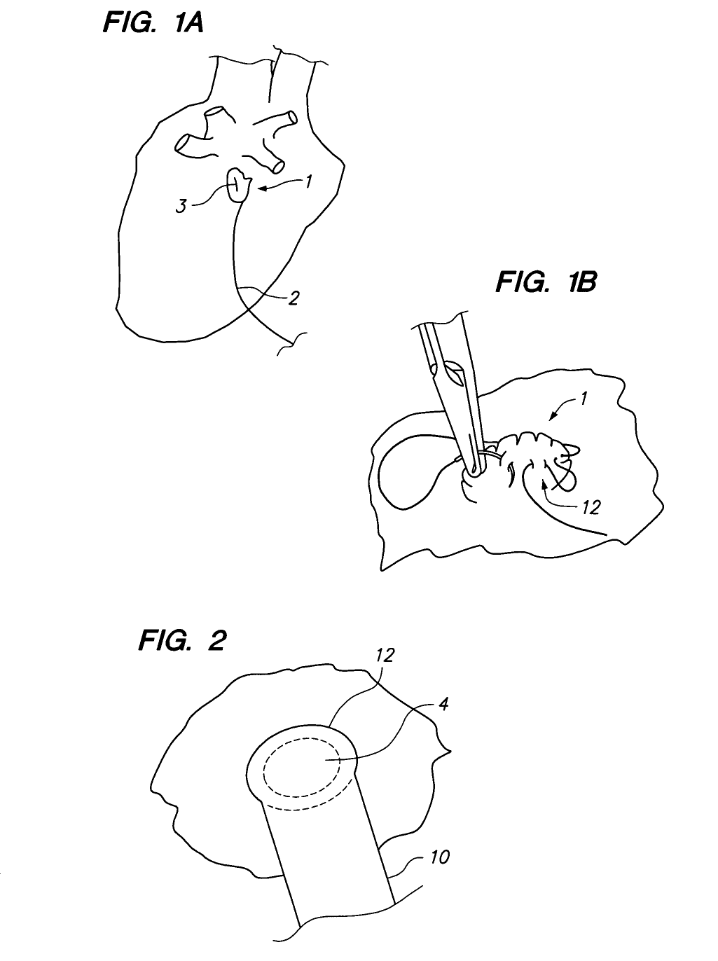

A particularly useful and relatively less invasive method of performing atrial ablation involves access through a small thoracotomy. For example, a small incision (e.g., about 2 cm in length, although this length may vary) is made between the ribs of a patient, typically along a mid-clavicular line, around the third intercostal space (between third and fourth ribs. A surgical cutting instrument is introduced through this opening to incise or open the pericardium, after which the atrial appendage is located using an endoscope and an endoscopic instrument such as a surgical grasper (e.g., a 5 mm endoscopic grasper) or other endoscopic instrument. A purse-string suture 2 is placed around a section of the free border of the atrial appendage 1 (as illustrated in FIG. 1A) after which an incision 3 is formed through the atrial appendage 1, to form an opening large enough to insert an ablation device therethrough. FIG. 1B shows an enlarged, detailed portion of FIG. 1A showing the formation of the purse-string suture 2 being formed in the atrial appendage 1 in more detail. For example, incision 3 may be made large enough to accommodate a port of about 7 to about 25 mm in diameter through which an ablation device may be inserted. If used, the port may contain a hemostatic seal, as will be described in more detail below, to seal against blood loss between the port and the ablation device. Pressure may be applied by tightening the purse string suture to close off the opening so that no or only a minimal amount of blood is released therefrom during removal of the port (or ablation device, if no port is used). The purse-string suture may be placed first, followed by incision of the atrial appendage, to allow insertion of an instrument. Alternatively, a surgical clamp may be placed across the base of the appendage; in this case, the incision may be made before placement of the purse-string suture, as the clamp provides hemostasis. If a port of delivery guide is not used, the purse string suture may be tightened after insertion of the ablation device to prevent blood loss around the instrument and through the opening in the atrial appendage during the length of the procedure. Although illustrated with regard to a left atrial procedure, a similar procedure may be performed through the right atrial appendage to perform ablation procedures endocardially in the right atrium.

The ablation instrument may then be manipulated to directly position an ablation element against one or more locations of the endocardium to be ablated during the procedure. For example, FIG. 2 illustrates a cutaway view of an ablation instrument 10 having been inserted through an atrial appendage according to the technique described above, and then manipulated/maneuvered to cannulate a pulmonary vein ostium 4. An ablation element 12 is positioned to circumscribe the ostium, where a lesion is then generated by ablating cardiac tissue surrounding the ostium.

The present techniques not only negate the need for opening the chest and the heart for performing the ablations, but also do not require the heart to be stopped and the patient to be placed on bypass. Additionally, when performing ablation procedures in the left atrium, these procedures negate the need of forming a trans-septal opening, as is required by percutaneous catheter-delivered systems.

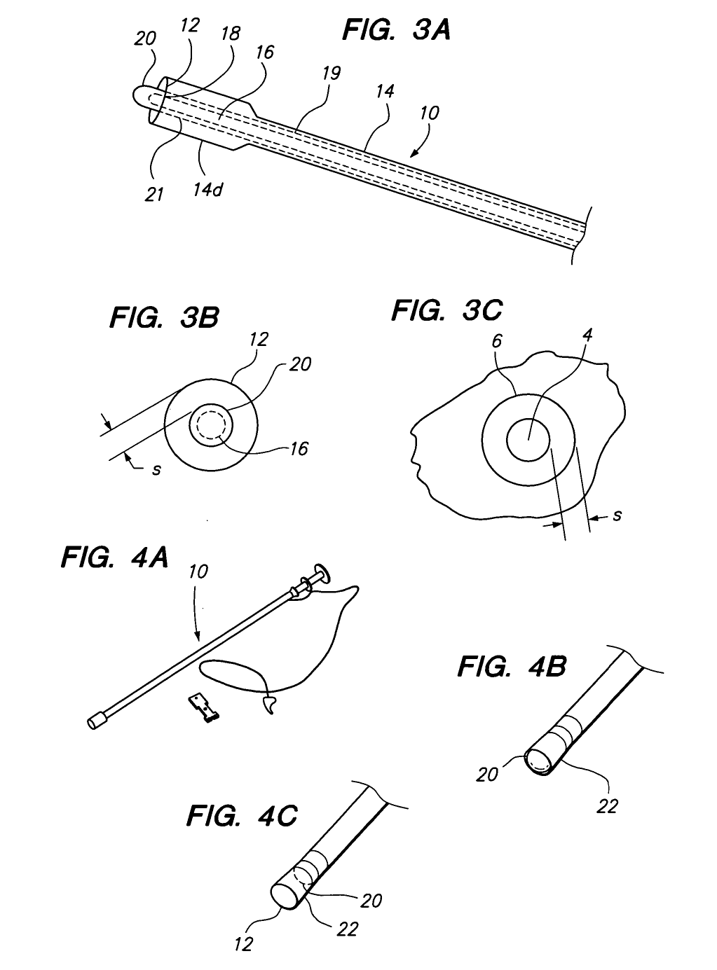

FIG. 3A shows a perspective view and FIG. 3B shows an end view of an example of an ablation instrument that may be used to practice the techniques described above. Ablation element 10 includes an elongated tube or shaft 14 that houses or surrounds an endoscope 16 (e.g., a rigid tube/telescope having a diameter of about 5 mm and length of about 25-40 cm). Such endoscopes are available from various companies, including Olympus (Japan), and Stortz and Scholly (Germany).

Tube or shaft 14 is typically rigid to provide the best maneuverability, once instrument 10 has been inserted into the area to perform the surgical techniques, for guiding the distal end of instrument 10 to the desired locations(s) to perform the procedures. A rigid tube or shaft is generally preferred for the techniques involving insertion of instrument 10 through an atrial appendage, as described above. For example, a rigid tube 14 makes it easier to guide the tip and ablation element of the ablation instrument to each pulmonary vein ostium or to any desired location within the atrium where it is desired to form an ablation.

However, the distal end portion may be formed to be articulating, to provide a greater range of motion in directing the distal end of the instrument to the target site. Further alternatively, tube or shaft 10 may be made flexible or malleable for situations in which a flexible endoscope is inserted therein and where it would be advantageous for the particular application or technique being practiced.

A light emitter or source 18 is provided in the distal end portion of instrument 10 to direct light out of the distal end so that the operator may visualize the position of the distal end in the surgical site by viewing through the endoscope 16. Thus, a surgeon or operator may directly view the positioning and movements of the distal end of instrument 10 from outside the patient, without the need to resort to any indirect visualization or sensing techniques for positioning, and this greatly increases the accuracy and precision of placement of instrument 10 for performing ablation. A power supply line 19 may be connected to light source 18 and extend proximally out of the instrument to be connected to an external power source.

An atraumatic, transparent tip/lens 20 is provided at the distal end of instrument 10. Tip/lens 20 enables direct viewing of the surgical site through endoscope 16 (e.g., direct visualization of the endocardial surface and particularly the pulmonary vein ostia within the left atrium when performing ablation endocardially from within the left atrium).

Tip 20 is formed in a hemispherical configuration as shown in FIG. 3A, but may be formed in other blunt shapes so as to prevent injury to the endocardial tissue upon contact therewith. Endoscope 16 may be axially translatable with respect to tube 14 so as to change the distance of the scope from the distal end of instrument 10, and tip 20 may be configured to allow the scope to be slid within the confines of tip 20, as shown in FIG. 3A.

The distal end portion 14d of tube 14 as shown in FIG. 3A has a larger outside diameter than the remainder of tube 14. Distal end 14d is made larger to enable the mounting of ablation element 12 in a location that is spaced away from the perimeter of tip 20 (see FIG. 3B). This configuration ensures that a lesion will not be formed in a pulmonary vein ostium, as will be described below. Ablation element 12 as shown is a circumferential electrically conducting element that is mounted around the circumference of the distal end of distal end portion 14d, as shown, and is connected to a pair of leads or wires 21 that extend proximally through tube 14 and out of device 10 to be connected with a source of radio frequency energy in this case. For example, wire 21 may be connected outside of the instrument 10 and patient to an Rf generator (e.g., such as those available Valleylab, Farmingdale, N.Y.). However, neither instrument 10 shown in FIGS. 3A-3B, nor any of the other ablation instruments described herein are limited to the use of Rf ablation, Rf ablation elements, or circumferential elements. Various types of ablation elements may be employed, including radiofrequency (RF), microwave, ultrasound, heated fluids, cryogenics and laser. Further, rather than a full circumferential element, an arc-shaped or single point element may be provided and instrument 10 may be rotated if a circumferential lesion is desired to be formed.

In one example of the use of the ablation instrument of FIG. 3A, instrument 10 is inserted through a small thoracotomy and through the left atrial appendage according to the techniques described above, in order to establish pulmonary vein exclusion. By viewing through endoscope 16, the surgeon or operator is able to visually direct the distal end of instrument 10 within the left atrium to guide it to the desired surgical targets. In this case, the operator guides the distal tip into an ostium of a pulmonary vein to a position as illustrated in FIG. 2. Distal tip/lens 20 is sized so that the outside diameter thereof approximates the inside diameter of the ostium of the pulmonary vein. By providing lens 20 to be about the same size as the ostium, lens 20 is inserted into the ostium and approximated therewith, which enables the surgeon/operator to clearly see the ostium when viewing through endoscope 16.

As noted above, ablation element 12 (ring, single element, arc, or whatever configuration) is positioned radially outside of the circumference of lens 20 and spaced by a distance “s” to ensure that it does not contact the ostium. Once cannulated, so that lens 20 approximates the ostium as shown in FIG. 2, energy (or any other ablation expedient, e.g., chemical) may be applied through ablation element 12 to create a lesion 6 where element 12 approximates the endocardium. The term “approximate” is used here to denote that the ablation element 12 either contacts the endocardium (as in the case where the ablation element delivers Rf energy, for example) or is placed closely adjacent to, but not contacting the endocardium, at a distance across which ablation energy can be effectively delivered to the tissue/endocardium to generate a lesion. For example, and ablation element that delivers microwave energy may be spaced slightly from directly contacting the tissue to be ablated by a dielectric medium. A distal portion of the device (such as lens 20, in this example) may be configured so that when it contacts tissue, the ablation element is separated from the tissue by a desirable distance to optimize the formation of the lesion. The surgeon/operator can view the ostium in real time as the lesion is being created external (i.e. radially external) to the ostium, thereby ensuring that no portion of the lesion created intersects with the ostium.

In this example, Rf energy was applied through a ring-shaped or circumferential ablation element 12 to form lesion 6. After completion of the formation of the lesion 6, instrument 10 is removed from the site leaving a lesion 6 circumferentially spaced from ostium 4 as shown in FIG. 3C. Such removal may also be performed intermittently to test the sufficiency of the lesion formed (e.g., to determine whether the lesion has been established fully transmurally or established to an extend sufficient to adequately block the conduction of signals originating in the pulmonary vein or ostium that the lesion surrounds) and the instrument may be reinserted according to the techniques described above to further the lesion formation process when it is determined that the lesion has not yet been sufficiently formed. Tissue temperature may be measured to determine the degree of transmural heating, which can be used to judge the sufficiency of a lesion that has been produced. Additionally or alternatively, tissue electrical impedance may be measured between the endocardial (inner) and epicardial (outer) surfaces of the tissue. Further, a pacing electrode may be placed inside a pulmonary vein, and inability to achieve successful pacing of the heart, is another indictor that sufficient ablation (a successful lesion) has been performed.

The above described procedures may be repeated for each of the remaining three pulmonary veins/pulmonary vein ostia to establish pulmonary vein exclusion, by creating atrial lesions 6 in the atrial tissue surrounding each of the pulmonary veins/pulmonary vein ostia. The creation of lesions within the pulmonary ostia has been reported to be linked with the development of pulmonary vein stenosis. Thus, the present invention ensures that lesions are not created within the pulmonary vein ostia, but only in atrial tissue external to the ostia.

Tip/lens 20 is substantially rigid and has fixed dimensions. Because the sizes of pulmonary vein ostia may vary from patient to patient, and further since sizes of pulmonary vein ostia within the same patient often vary, the configuration of FIG. 3A may require that several ablation instruments 10 be made available, each with different tip 20 sizes, to accommodate the variation in ostia that may be encountered. This is so because the tip 20 must conform quite closely to the inside diameter of the ostium to be viewed. If tip 20 is too large, then it cannot be inserted into the ostium and is of limited value in carrying out ablation procedures of this type. If tip 20 its too small, then all or a portion of the tip will not engage the ostium properly for viewing and blood flow will cover all or a portion of the circumference of tip 20 so that the ostium cannot be clearly viewed.

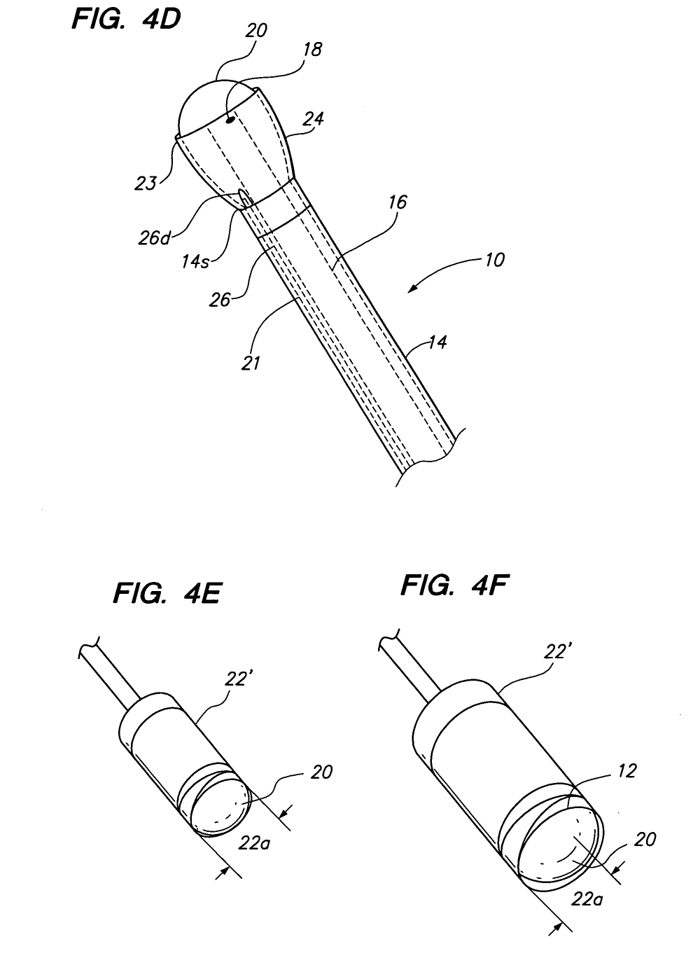

FIGS. 4A-4D show another example of an ablation instrument 10 according to the present invention. Similarly to the instrument of FIG. 3A, ablation instrument 10 in FIG. 4A includes an elongated tube or shaft 14 that houses or surrounds an endoscope 16. Tube or shaft 14 is typically rigid to provide the best maneuverability, once instrument 10 has been inserted into the area to perform the surgical techniques, for guiding the distal end of instrument 10 to the desired locations(s) to perform the procedures, although like the example of FIG. 3A, the distal end portion may be formed to be articulating, to provide a greater range of motion in directing the distal end of the instrument to the target site. Further alternatively, tube or shaft 10 may be made flexible or malleable for situations in which a flexible endoscope is inserted therein and where it would be advantageous for the particular application or technique being practiced.

A light emitter or source 18 is provided in the distal end portion of instrument 10 to direct light out of the distal end so that the operator may visualize the position of the distal end in the surgical site by viewing through the endoscope 16. Thus, a surgeon or operator may directly view the positioning and movements of the distal end of instrument 10 from outside the patient, without the need to resort to any indirect visualization or sensing techniques for positioning, and this greatly increases the accuracy and precision of placement of instrument 10 for performing ablation. A power line 19 may be connected to light source 18 and extend proximally out of the instrument to be connected to an external power source.

An atraumatic, transparent tip/lens 20 is provided at the distal end of instrument 10. Tip/lens 20 enables direct viewing of the surgical site through endoscope 16 (e.g., direct visualization of the endocardial surface and particularly the pulmonary vein ostia, cardiac valves, papillary muscles, cordae tendonae, septal defects, etc. when used in the endocardial environment. Tip 20 is formed in a hemispherical or “dome” configuration as shown in FIG. 4A, but may be formed in other blunt shapes so as to prevent injury to the endocardial tissue upon contact therewith. Dome 20 is formed as a rigid, fixed structure, such as from a transparent glass or rigid polymer material, but may alternatively be formed from a flexible transparent material, and may be adapted to have a variable size as discussed below.

Unlike the example in FIG. 3A, instrument 10 in FIG. 4A is provided with a sliding ring 22 at the distal end thereof. Sliding ring is configured to slide with respect to and over the distal end and distal tip 20 of instrument 10. FIG. 4B shows sliding ring 22 in a retracted position so that tip/lens 20 protrudes distally therefrom, similar to the configuration of instrument 10 in FIG. 3A. This configuration of instrument 10 is useful for manipulating and positioning the instrument 10 in the desired surgical target area, as when tip/lens 20 protrudes distally from sliding ring 22, a relatively better and wider angle of view is provided to the endoscope 16. Once a surgical target is visually identified and instrument 10 is placed on the target (e.g., so that lens 20 contacts or at least points at the target of interest) sliding ring 22 is slid distally into the position shown in FIG. 4C. The distal end of sliding ring 22 has at least one ablation element 12 mounted thereon. Insulation (electrical insulation) 23 (such as a polymeric, ceramic or glass layer or coating, for example) may be provided around the perimeter of ablation element 12 to prevent energy loss to circulating blood. Insulation may also be applied to the inside of ring/ablation element 12 to prevent energy loss to saline, as it passes over the ablation element 12. A dielectric layer may be provided distally, as in the case of use of a microwave ablation element, to provide the desired spacing between the ablation element and tissue upon contacting the tissue with the distal end of the ring. Sliding ring 22, when contacted against the myocardial surface at the surgery target, establishes a working space or working field within the confines of ring 22 which can be directly observed via endoscope 16 through lens 20. Firm contact between ring 22 and the endocardial surface stabilizes the working field. Positive pressure irrigation (e.g., such as by saline) may be applied to the working space by delivering the irrigation fluid through at least one conduit provided within the sliding ring and extending proximally out of instrument 10 (see FIG. 4D).

Alternatively, saline can be flowed through the annular space between tubes, without a conduit directing it, or by a separate lumen or side channel. The irrigation maintains a clear visible field in the working space so that the ablation can be performed under real time, direct visual observation. In this example, electrical energy is applied to ablation element 12 to electrically isolate the tissue inside ring 22, by forming a lesion, such as by the application of Rf energy through ablation element 12. Alternatively, the sliding ring may not be electrically conductive at all, but the saline can act as the electrical conductor to apply the ablation energy to the tissue, as described further below. Thus, this configuration is flexible in its application to ablation procedures, as tip 20 need not be inserted into an ostium to form an ablation. Rather, since sliding ring slides to extend distally of tip 20, ablation element 12 may be approximated to any surface that is desired to be ablated.

In the example shown, two concentric tubes 14 and 13 are provided with inner tube 13 being longer that outer tuber 14. Sliding ring 22 is attached to outer tube 14, and endoscope 16 is inside of inner tube 13. An annular space existing between inner tube 13 and outer tube 14 is used for saline irrigation and houses a conductive wire (which is electrically connected to ring 22 when ring 22 is conductive, and otherwise transmits/conducts ablation energy directly to the saline flowing thereover when the saline is used to apply the ablation energy. Relative motion of the ring 22 and lens 20 is achieved by telescoping the inner and outer tubes (i.e., axially sliding the tubes with respect to one another). The saline may be delivered under pressure sufficient to displace the walls of balloon 20 to make a pathway through which the saline flows. There is also a natural “leak” or pathway provided by the interface between the balloon and the last (innermost) winding of the coil of ring 22. Alternatively, an actuation rod or wire 26 may be provided through tube 14 for sliding actuation of sliding ring 22 from a proximal location outside of instrument 10. The distal end 26d of rod or wire engages sliding ring 22 and slides in a slot 14s in tube 14 during sliding maneuvers of sliding ring 22. Various other mechanical arrangements for relatively displacing ring 22 relative to lens/tip 20 may be equivalently provided, as would be apparent to one of ordinary skill in the art. Endoscope 16 may be axially translatable with respect to tube 13 so as to change the distance of the scope from the distal end of instrument 10, and tip 20 may be configured to allow the scope to be slid within the confines of tip 20.

Ablation element 12 as shown is a circumferential electrically conducting element that is mounted around the circumference of the distal end of sliding ring 22, as shown, and is connected to a pair of leads or wires 21 that extend proximally through tube 14 and out of device 10 to be connected with a source of radio frequency energy in this case. For example, wires 21 may be connected outside of the instrument 10 and patient to an Rf generator. However, other types of ablation elements may be employed, including monopolar radiofrequency (RF), microwave, ultrasound, heated fluids, cryogenics and laser. Further, rather than a full circumferential element, one or more arc-shaped or single point elements may be provided and instrument 10 may be rotated if a circumferential lesion is desired to be formed.

As noted earlier, tip 20 may be formed as a transparent balloon, such as from a transparent elastomer, for example. With such a configuration, sliding ring 22 may be modified so as to be formed as a spiral conductor ring 22′ as shown in FIGS. 4E-4F, for example. With this arrangement, ring 22′ may be a spirally formed, electrically conductive spring having a preloaded small or contracted diameter. In this case, by inflating tip 20 inside of ring 22′, the expanding tip/balloon 20 drives ring 22′ to a larger/expanded configuration (FIG. 4F), as the coils of the ring slide with respect to one another as the inside diameter of ring 22′ is driven larger. As the balloon is being deflated, ring 22′ contracts (as shown by the smaller diameter 22a in FIG. 4E), following the contraction of the balloon as it is in this case driven by the preload on the spiral conformation of ring 22′. Ring 22′ may be made from spring metal, with both inside and outside of the metal being coated with a lubricious insulator, such as PTFE, nylon, PEEK, or the like. In this case the conductive surface (i.e. distal edge of ring 22′) acts as the ablation element 12. The laminate insulation insulates against both the blood flowing outside over ring 22′ as well as the saline flowing inside the ring 22′. Ring 22′ may also be translated relative to expandable tip 20 by any of the methods described with regard to FIGS. 4A-4D above. Alternatively, ring 22′ may be translated relative to tip 20 by telescoping the coils or the ring 22′ (like a Chinese yo-yo). Thus, this arrangement enables the user to vary the size of the working field around which to ablate and establish a lesion. This feature may be useful for creating lesions around pulmonary vein ostia of various sizes, as well as for other applications where the surgical target size varies. Expandable lens 20 maintains the capability of real time viewing of the procedure by endoscope 16.

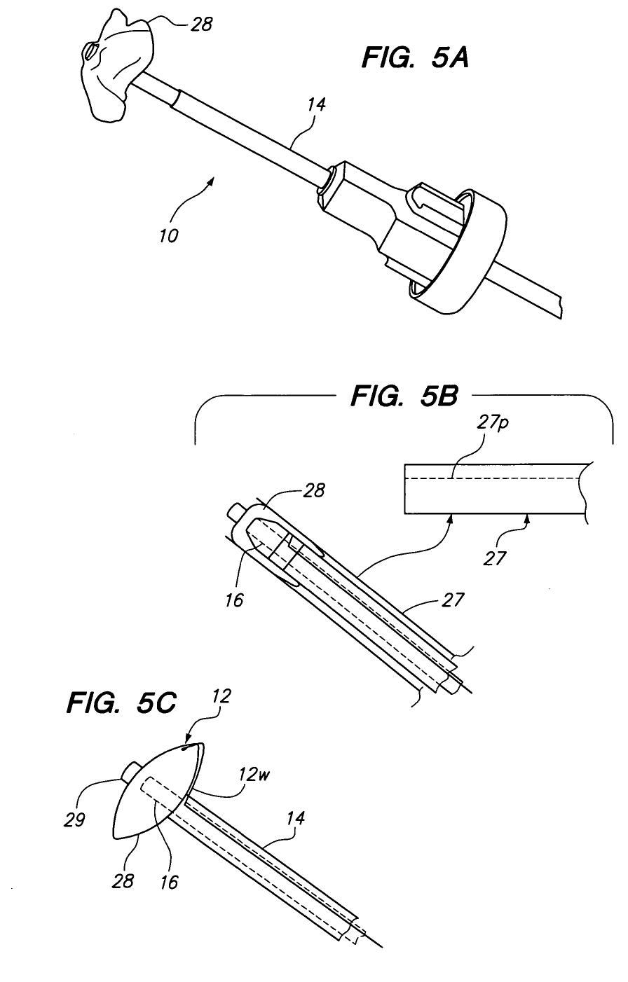

FIG. 5A shows another example of an ablation instrument 10 having a working end portion that is adjustable in size. Instrument 10 includes a tube or cannula 14 that is rigid, or may be malleable in some situations, just as discussed with the above embodiments. A transparent, flexible, generally inelastic balloon 28, which may be made of polyethylene, polyurethane, polyvinyl chloride, polyethylene terepthalate, or the like, is mounted on the distal end of tube 14. Tube 14 accommodates an endoscope 16 within its lumen, in the same manner as described above (not shown in FIG. 5A). A luer port 30 is provided in a proximal end portion of instrument 10 and connects with a lumen that passes internally of tube 14 and fluidly connects to balloon 28.

When deflated, balloon 28 may be gathered about the distal end portion of tube 14 to provide a smaller diameter profile that facilitates insertion of the distal end portion of instrument 10 (requiring only a relatively small thoracotomy (about 2 cm)) through an opening in the patient and through the atrial appendage. As a vacuum is drawn on the balloon 28, balloon 28 may be wrapped around the cannula/tube 14 and heated gently to cause the balloon to remain in a small profile. Alternatively, a relatively thin (e.g. about 0.002″ thickness) plastic sheath 27 may be pulled over the wrapped balloon, as illustrated in FIG. 5B. Plastic sheath 27 may have a set of longitudinal perforations 27p running over its length, allowing it to be peeled away upon balloon inflation. After inserting the distal end portion of instrument into the surgical working space (e.g., after passing the distal end portion through the atrial appendage) balloon 28 may be inflated by connecting a fluid source (such as a saline-filled syringe, for example) to luer port 30 and delivering the fluid under pressure to balloon 28 to inflate it. In the inflated configuration, the transparent tip/balloon 28 has an outside diameter substantially larger than an outside diameter of tube 14 as shown in FIG. 5B. For purposes of pulmonary vein exclusion, the diameter of balloon 28 is substantially greater than the inner diameter of the pulmonary vein ostia, making it impossible for the balloon to enter the ostium of a pulmonary vein to be excluded.

A flexible ablation element 12 is attached to the distal face of balloon 28 (see FIG. 5C) and connected with a source of ablation energy (that may be located proximally of the device, outside the patient, for example) via wire conductor 12w. Ablation element 12 may be adhered to the surface of balloon 28, for example, using room temperature vulcanization (RTV) silicone rubber, or the like. Ablation element 12 may be connected with one or more power supply lines, as discussed with regard to above examples (although power supply lines may run external to tube 14 and may supply one of a variety of energy types, including radiofrequency energy, microwave energy, laser energy, electrical resistance heating, cryogenics, ultrasonic energy, etc. Ablation element 12 may be made from a variety of different materials, the choice of which also may vary depending upon the type of energy to be delivered to perform the ablation. For example, an Rf element may be stainless steel, while an element for supplying laser energy may be a fiberoptic cable (silica), and so forth. Further, a dielectric material may be mounted on a distal side of ablation element 12 to provide proper spacing for delivery of microwave ablation energy and/or a distal extension may be provided to extend distally beyond the ablation element to establish a proper separation distance between a microwave ablation element and the tissue upon contacting the tissue with the distal extension.

The distal end of endoscope 16 resides inside balloon 28, thereby allowing visualization of the surgical field (e.g., endocardial surface) that contacts the distal face of balloon 28. An outline of ablation element 12 is also visible through balloon 28 via endoscope 16. In one example of use, instrument 10 is manipulated from outside the patient to move inflated balloon 28 along the endocardial surface of the left atrium until the operator visually verifies that ablation element 12 has encircled a pulmonary vein ostium. In this example, ablation element is of a circular, oval or other encircling configuration with dimensions sufficient to surround a pulmonary vein ostium without intersecting with the ostium. Once the operator has visually verified that ablation element 12 has encircled the ostium and does not contact or intersect the ostium at any location along its perimeter, ablation element 12 is energized to perform the ablation of the endocardial tissue surrounding the ostium while the operator visually observes the ablation through endoscope 16.

Endoscope 16 may be moved axially within balloon 28 (i.e., with respect to the longitudinal axis of tube 14) to change the visual field, e.g., allowing visualization of a narrow or wide field of view as needed. For example, the distal tip of endoscope 16 may reside close to the distal face of balloon 28 as instrument 10 is moved around the left atrium to identify a pulmonary vein orifice/ostium. Once the ostium has been located and identified, instrument 10 is held stationary and endoscope 16 is retracted proximally with respect to balloon 28 (but not so far as to retract the distal tip of endoscope 16 completely out of balloon 28) to provide a wide viewing angle to allow visualization of ablation element 12 and atrial endocardium surrounding the pulmonary vein ostium. Using this viewing angle, instrument 10 may then be finely adjusted to properly position ablation element 12 so that the pulmonary vein ostium is centered within the surrounding ablation element 12, or at least to ensure that ablation element 12 does not contact or intersect with the pulmonary vein ostium. The wide viewing orientation of endoscope 16 is maintained during performance of the ablation, so that ablation element 12 and the progression of the formation of the lesion during the ablation may be viewed in real time by the operator through endoscope 16.

Balloon 28 as shown in FIGS. 5A and 5B has a smooth distal face. Alternatively, balloon 28 may be formed with a protruding nipple 29 on its distal face, as shown in FIG. 5C. Nipple 29 may be used to cannulate a pulmonary vein ostium, thereby making it easier to hold balloon 28 (and ablation element 12) centered in place about the pulmonary vein ostium while ablation is performed. The outer diameter of nipple 29 is formed to be smaller than the inside diameter of the pulmonary vein being excluded, and the outer diameters of balloon 28 and ablation element 12 are substantially greater than the inner diameter of the pulmonary vein ostium, as noted above.

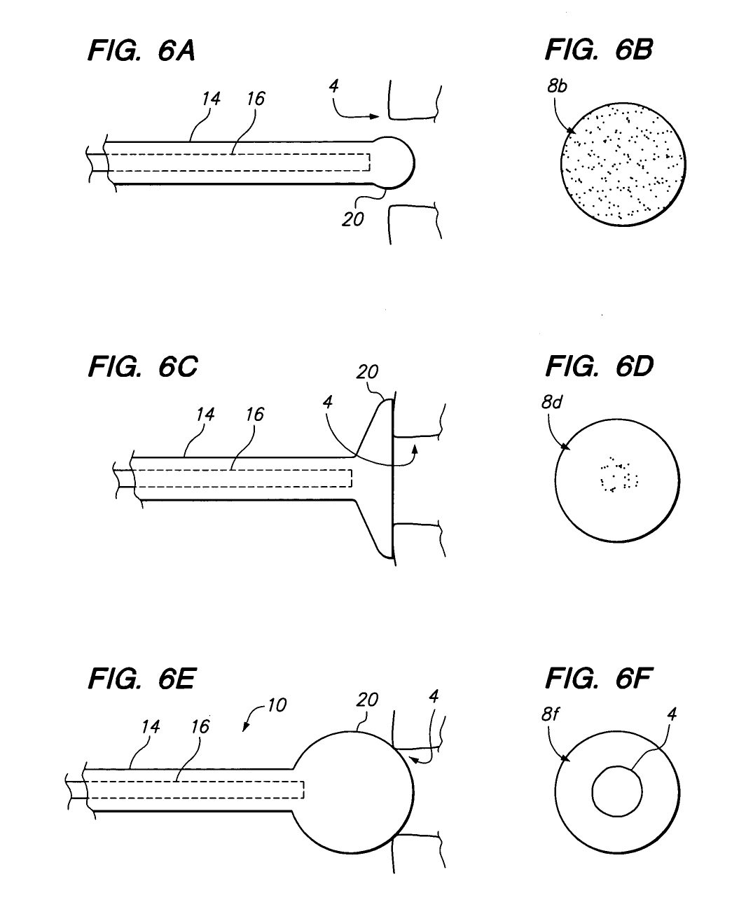

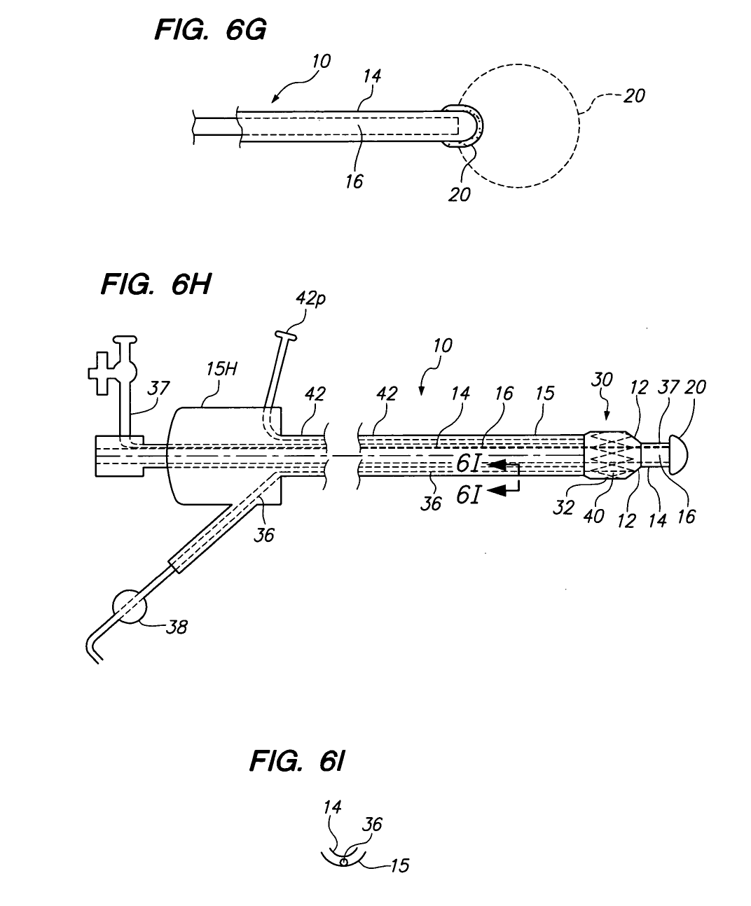

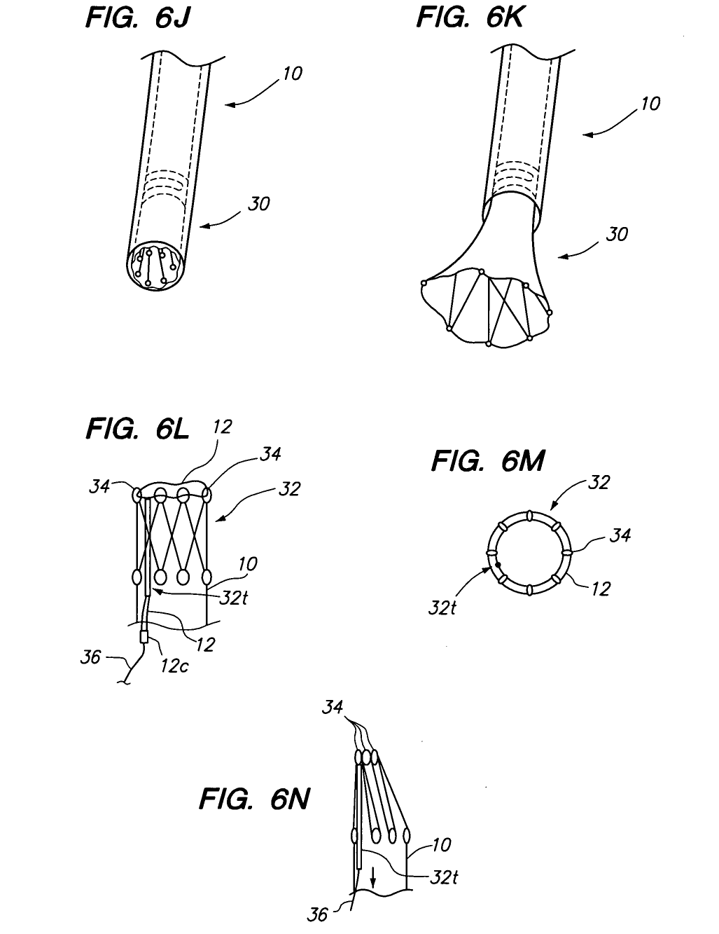

As already noted, pulmonary vein ostia diameters vary: the inside diameters of human pulmonary vein ostia vary generally from a range of about 11 mm to about 20 mm, and sometimes even up to about 25 mm. In order to visualize an ostium, the distal tip/lens of an ablation instrument should have an outside diameter that approximates the inside diameter of the ostium to be viewed, to provide clear visualization. If the tip/lens is too small, visualization can be obscured by blood flow. For example, use of an ablation instrument 10 having a spherical, hemispherical or dome-shaped tip 20 with an outside diameter of 10 mm to attempt to visualize an ostium having an inside diameter of 20 mm (as illustrated in FIG. 6A) permits blood to flow between the walls of the ostium 4 and the walls of the tip 20 resulting in an obscured view 8b of the blood flowing over the walls of tip 20 as illustrated in FIG. 6B.

Further, the distal tip/lens of an ablation instrument used to visualize a pulmonary vein ostium should be relatively rigid in order to provide a clear view of the ostium. A tip that is excessively soft or flexible tends to “flatten out” or deform as it is pressed against the atrial wall. Thus, for example, use of an ablation instrument 10 having a spherical, hemispherical or dome-shaped elastic tip to attempt to visualize an ostium results in the tip deforming as the instrument 10 is pressed against the atrial wall to approximate an ablation element against the endocardium, as illustrated in FIG. 6C. The flattened or deformed tip 20 covers over the pulmonary vein ostium 4 rather than approximating it, resulting in an image 8d of a spot of blood on a white background of atrial tissue (endocardium) as illustrated in FIG. 6D, with no direct visualization of the edge (border) of the ostium 4.

Accordingly, the present invention provides a tip 20 with sufficient structural rigidity needed to cannulate the pulmonary vein ostium 4 and with a size (outside) diameter sufficient to approximate the inside diameter of the ostium, that is, the outside diameter is not sufficiently greater than the inside diameter of ostium 4 to prevent insertion of tip 20 into ostium 4, but is not so small as to permit blood flow between tip 20 and the walls of ostium 4 to obscure the field of view. In addition to the disadvantage explained above, if tip 20 is too flexible, slight movement of instrument 10 or application of force may bend tip 20 and cause it to be displaced out of the pulmonary vein ostium 20. A spherical or hemispherical tip with sufficient rigidity straddles the pulmonary vein ostium to provide a clear endoscopic view of the outline or border of the ostium.

FIG. 6E illustrates an example of use of an ablation instrument 10 according to the present invention to view a pulmonary vein ostium in preparation for carrying out an ablation technique as described above. Spherical tip 20 is of sufficient size to approximate ostium 4 and has sufficient rigidity to straddle ostium 4 so that the approximation against the ostium 4 provides a clear visualization of the ostium edge or border through endoscope 16, as illustrated in FIG. 6F (see view 8f, where the border of ostium 4 is clearly visible).

It is desirable to form tip 20 as an elastomeric balloon attached to the distal end of tube 14, to enable the tip to be inflated/expanded to the dimensions and rigidity desired for visualization of the ostia, as described above, while also permitting tip 20 to be deflated/contracted during insertion/delivery of the distal end portion of instrument 10 to the surgical target site. The balloon may be glued directly to the cannula, using epoxy, ethyl cyanoacrylates (such as LOCTITE 4011, for example) or light curing adhesive, for example. A suture winding (e.g., a silk suture winding, or the like) may also hold the balloon in place, with adhesive coating the suture winding. A heat shrink plastic tube may be shrunk over the glued balloon and cannula interface to provide further reinforcement. This allows tube 14 to be made with a significantly smaller outside diameter as well. The deflated tip 20 fits snugly on tube 14 to minimize the profile of the distal end portion for delivery purposes. For example, it is desirable to provide tube 14 with a relatively small outside diameter (typically about 7 mm to about 10 mm) to facilitate insertion through a limited incision in the atrial appendage, while providing tip 20 the capability of expanding to an outside dimension/diameter up to about 20 mm, or up to about 25 mm. It is difficult to pass an instrument having a tube diameter of 20 mm through the atrial appendage, and also more difficult to maneuver the instrument if indeed there is success with passing the instrument through the atrial appendage.

FIG. 6G illustrates an example of an ablation instrument 10 having an expandable distal tip 20, showing both the deflated state of tip 20 and an inflated configuration (in phantom). Tip 20 is formed of an elastomeric material, such as silicone rubber, or other elastic material including latex rubber, C-FLEX® (a thermoplastic elastomer of styrene-ethylene-butylene (SEBS) modified block copolymer with silicone oil), polyurethane, or other biocompatible thermoplastic elastomer that is sufficiently transparent. Following insertion of the distal end portion of instrument 10 into the surgical working space (e.g., following insertion of the distal end portion through the atrial appendage) balloon 20 is inflated to about 300% to 500% elongation of the balloon material, by delivering fluid (e.g., saline) to balloon 20 in a manner such as described above with regard to the example of FIGS. 5A-5B. The surface tension in balloon 20 so inflated/expanded causes inflated balloon 20 to be sufficiently rigid to perform the task shown in FIG. 6E, thereby providing excellent visualization of the pulmonary vein ostium.

Referring to FIG. 6H, an outer tube 15 (e.g., having an outside diameter of about 9 to about 12 mm) is provided coaxially over tube 14, to which an expanding member 30 is mounted. Expanding member 30 includes one or more ablation elements 12 mounted thereon to be used in ablating tissue when expanding member is in an expanded configuration. As shown in FIG. 6H, expanding member 30 is in a contracted or non-expanded configuration which is substantially tubular, to closely conform to outer tube 15 for purposes of minimizing the diameter of instrument 10 during delivery of the distal end portion to the surgical working space, such as to pass the distal end portion through the left atrial appendage and into the left atrium, for example. Tip 20 is also shown in the deflated/contracted configuration.

Expanding member 30 may be configured to form a substantially tubular or cylindrical shape when in a contracted configuration, such as shown in FIGS. 6H and 6J, for example, to closely conform to tubes 15 and 14 to minimize the cross-sectional area of instrument 10 during delivery. After placement into the surgical site, tip 20 may be expanded/inflated by delivering fluid under pressure through inflation tube 37, and expanding member 30 may be expanded to an expanded configuration, such as a substantially funnel-shaped configuration, to position ablation element(s) 12 radially outside of the circumference of tip 20 in its expanded configuration, as shown in FIG. 6K. The expandable member/ablation element may be slid distally with respect to the balloon by sliding tube 15 distally with respect to tube 14. A stopcock 39 or other shutoff or valving device is provided in line with inflation tube 37 and is closed to maintain the pressure within tip 20 after inflating. For example, the open, expanded (i.e., distal) end of expanding member 30 may have a diameter of about 30 mm to about 40 mm for an instrument having an expanded tip 20 with a diameter of about 20 mm.

Expanding member may include an expanding frame 32 which may be formed of a spring material, such as spring steel, Elgiloy® (a nickel-chromium spring steel alloy), or other spring metal that is biocompatible, or of a rigid plastic material such as polycarbonate, ULTEM® (amorphous thermoplastic polyetherimide), or similar material, or combinations of the previously listed metals and/or plastics. In one example, frame 32 may have a sinusoidal configuration, such as shown in FIG. 6L and may have eyelets 34 through which ablation element 12 may be threaded. Optionally, eyelets 34 may extend at an angle to, preferably perpendicularly to the longitudinal axis L of frame 32, as more clearly seen in the top view of FIG. 6M. Ablation element 12, in this example, may comprise a strand of electrically conductive wire and used to apply radiofrequency energy to the tissue to be ablated. Alternatively, other energy sources may be used to apply ablation energy, as with previous embodiments described. The wire forming ablation element 12 further extends (or is connected to another electrically conductive wire that extends) through a tubular extension 32t inside of frame 32 (see FIG. 6L) of a through lumen 36 and may be connected by connector 12c to a single pull wire/electrode 12e running through lumen 36, provided in tube 15 (see FIG. 6H and the partial sectional view of FIG. 6I), through lumen 36 and control knob 38 and further proximally to be connected with a source of ablation power, in this example, an Rf generator. The portion of the conductive wire lying inside through lumen 36 may be insulated (e.g., coated with an electrically insulating material such as plastic) so that only the wire 12 looped through eyelets 34 is electrically conductive.

When tension is applied to ablation element 12 by moving control knob 38 proximally with respect to handle 15H, the wire 12e extending through lumen 36 in tube 15 and connected to (or a part of) ablation element 12 cinches down the expanded frame 32 to its collapsed configuration as illustrated in the partial view of FIG. 6N. The ablation element runs through the eyelets, and the two tails of the ablation element course through the tubular extension. The two tails of the ablation element may then be attached by a connector 12c or soldered to a single wire conductor 36. When the wire 36 is tensioned, the ablation element 12 is drawn down the tubular extension, pulling the eyelets 34 together and cinching down the assembly.

Frame 32 may also be covered with a thin plastic or fabric sheet 40 to exclude blood and other fluids and/or tissues from the inner cavity formed by the covered expanding frame of expanding member 30. An irrigation lumen 42 may be provided within tube 15 to extend into the cavity formed by expanded expanding member 30 so that saline or other fluid may be fed from the proximally located irrigation port 42p and delivered into the cavity formed by expanding member 30 in the expanded configuration between tube 15 and tube 14. Such saline irrigation flushes blood from the interior of expanding member 30 to allow clear endoscopic visualization of ablation element 12 on the distal end of expanding frame 32 as it approximates tissue (e.g., atrial tissue) and performs the ablation.

Tissue blanching may be observed as the ablation proceeds, giving indication of the progress of formation of the lesion as it is formed. As atrial tissue is ablated, the resultant tissue desiccation causes blanching that is visible through the endoscope. In this way, visual analysis may be used to guide the adequacy of the ablation procedure. For example, when performing atrial ablation for treatment of chronic atrial fibrillation, a transmural ablation through the atrial tissue is desired to establish successful cessation of atrial fibrillation. Furthermore, extension of ablation energy beyond the heart tissue and into surrounding tissues is undesirable, and may cause complications, such as injury to the esophagus, among others. The endocardial surface of the atrium is generally composed of uniform muscle tissue (cardiac muscle), and there is no layer of fat present, in contrast to what is generally observed on the epicardial surface of the atrium. Consequently, energy applied to the surface of the endocardial tissue should conduct in all directions at approximately the same rate.

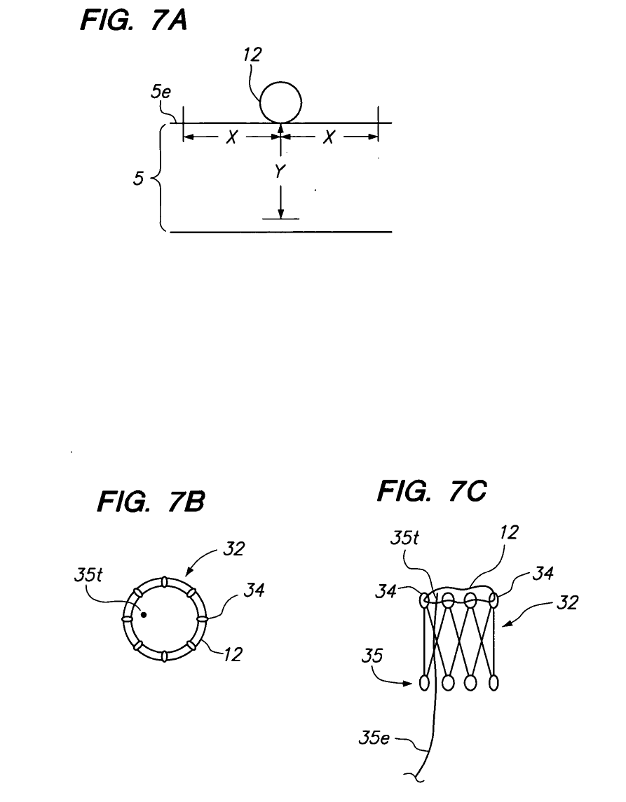

As illustrated in FIG. 7A, energy applied to the endocardial surface Se of the atrial wall 5 (or other heart wall) will travel depthwise (i.e., through the thickness of the wall 5) to approximately the same distance y as the distance x that the energy travels radially outward (i.e., along the endocardial surface) from the ablation element. Accordingly, given that the approximate thickness of the tissue wall being ablated is known (which can be an average generally known to those of skill in the art, or which may be measured using one or more techniques available in the art), a visual indicator 35 (which may also function for one or more other types of monitoring, such as does thermocouple 35, although an indicator which serves only as a visual indicator may be used alternatively) may be mounted so that the indicator tip 35t is positioned radially inside or outside of ablation element 12 by a distance approximately equal to the thickness of the wall of the tissue being ablated, as shown in FIGS. 7B and 7C. The indicator 35/indicator tip 35t may be made from plastic or metal (metal when necessary for performance of additional monitoring, such as when the indicator is also a thermocouple, for example), and may extend from one of the struts on frame 32. In the case where a thermocouple is employed, an insulated wire electrode 35e (FIG. 7C) connects the thermocouple tip 35t of the thermocouple to instrumentation for monitoring the thermocouple (not shown), in a manner known in the art, proximal of the ablation member 12 and generally outside of the body of the patient.

During use, when it is observed that the blanching of the endocardial surface reaches the extent of the visual indicator (and/or when some other indicator is observed, such as a predetermined temperature that is read by the thermocouple, which is believed to be in the range of about 50 to 60 degrees Centigrade), this is also indicative that the lesion/blanching (and/or other indicated condition, e.g., blanching temperature) has reached the epicardial wall of the tissue being ablated, so that a transmural ablation/lesion has been created.

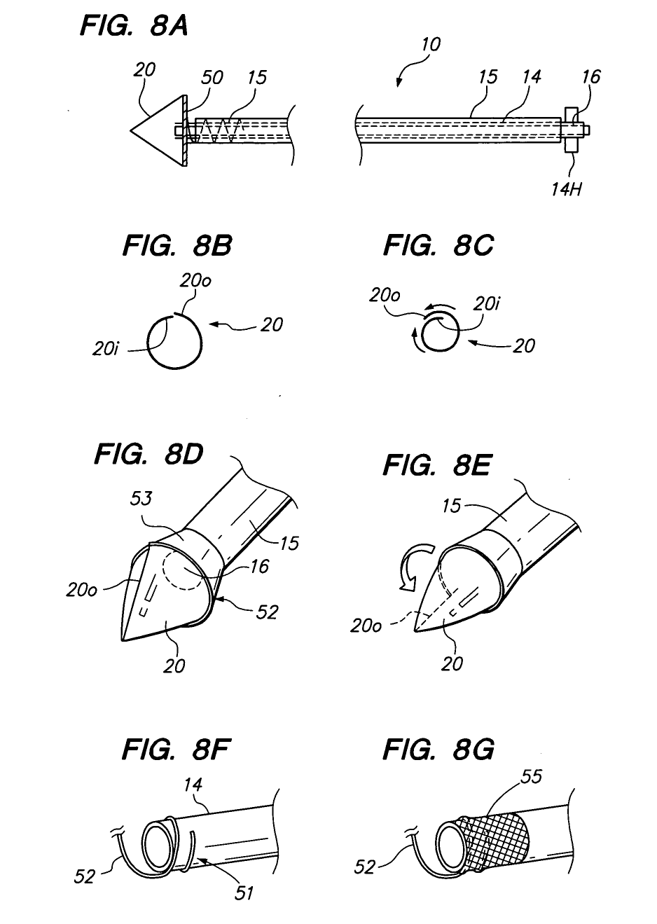

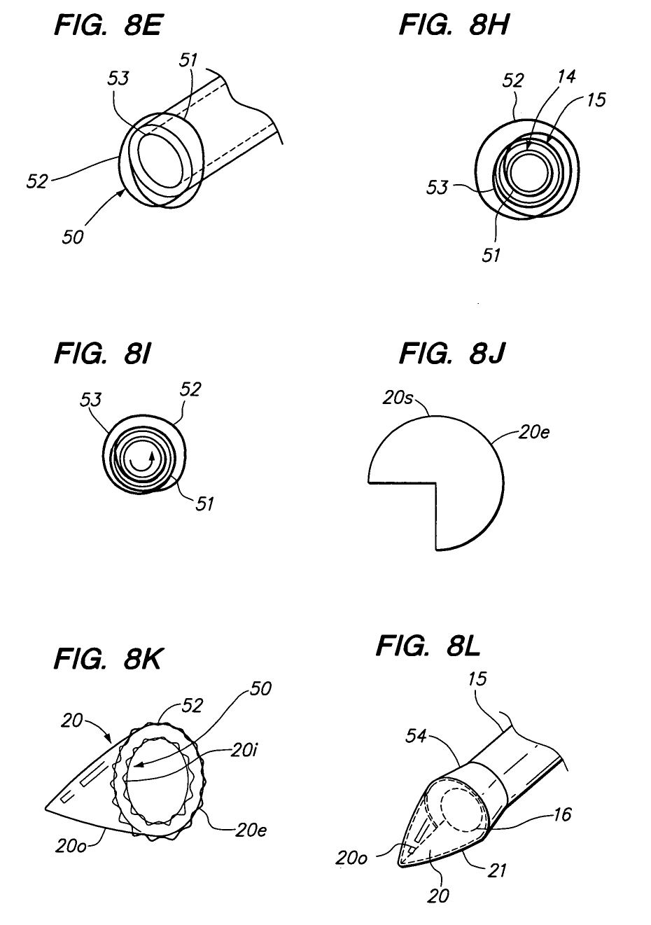

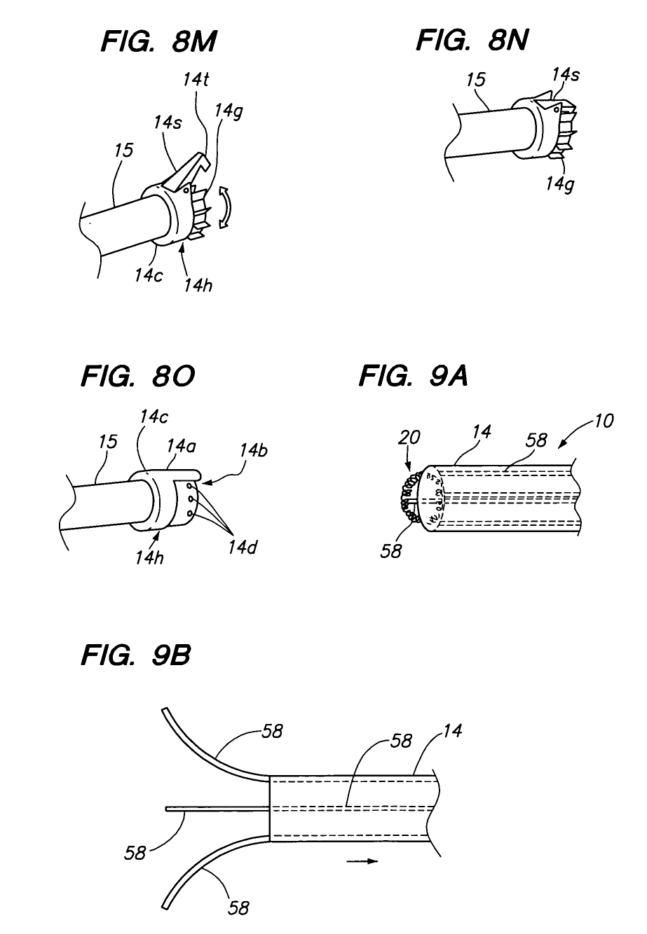

Referring now to FIG. 8A, an example of an ablation instrument 10 having a variable diameter tip 20 is shown. The instrument shown provides the user the ability to readily vary the size/diameter of tip 20 in real time, such as during ablation procedures. This is a particularly useful feature when performing more than one pulmonary vein exclusion, since the ostia dimensions typically vary among the four pulmonary veins of any given patient, as already noted. Instrument 10 includes tube 14 which houses endoscope 16 in the manner already described above. Outer tube 15 is provided coaxially over tube 14 and tubes 14 and 15 are configured to be rotated about their longitudinal axes with respect to one another. Tubes 14 and 15 are typically rigid, but may be malleable, such as described previously with regard to the above-described examples.

Tip 20 includes a conical lens in this example, formed by a sheet of overlapping, transparent, substantially rigid plastic. For example, the conical lens made be constructed from a sheet of polycarbonate, such as LEXAN® or the like, ABS polymer, such as LUSTRAN® or the like, for example. The sheet overlaps itself so that upon relative sliding of the outer overlapping edge relative to the underlying overlapped edge, the outside diameter of the conical lens increases or decreases, depending upon the direction of relative movement. FIGS. 8B and 8C illustrate this principle, where FIG. 8B shows the proximal end of the conical lens in its most expanded configuration, with outer edge 20o and inner edge 20i being close together while still maintaining an overlap. FIG. 8C shows the edges having been rotated, relative to one another in the directions of the arrows shown, which results in a reduction of the outside diameter of the tip 20. Reverse rotation re-expands the conical lens to increase the outside diameter of tip 20.

FIG. 8D is a partial perspective view of ablation instrument 10 showing tip 20 in a larger diameter configuration than that shown in FIG. 8E, where the configuration in FIG. 8D corresponds to what was described with regard to FIG. 8B and the configuration shown in FIG. 8E corresponds to what was described with regard to FIG. 8C.

To drive the relative movement of the outer edge 20o with respect to the inner edge 20i, a spring coil 50 is mounted at the distal end portion of instrument 10 between tubes 14 and 15. Coil 50 is preferably made from spring steel, Elgiloy® or other spring metal, but may be made from a polymer if it is not to be used to function also as an electrical or heat conductor, such as for purposes of an ablation element. Polymers that may be used to maintain the desired spring function include shaped carbon fiber rod, or braided tubing such as PEBAX® (polyether-block co-polyamide polymers) or HYTREL® rod (thermoplastic polyester elastomers), for example, so as to provide an inherent biasing force to its configuration. Typically, when no biasing is applied to coil 50 it is configured in the largest diameter position of the tip 20.

Coil 50 is fixed to tubing 14 at 51, as shown in FIG. 8E and coils around to an enlarged coil winding 52 that determines the outside diameter of tip 20, and continues with at least one reduced diameter winding to attach to tubing 15 at 53. Coil 50 may be fixed to tubing 14/15 by epoxy and shrink tubing 55 (FIGS. 8F and 8G), for example, wherein the shrink tubing 55 provides mechanical fixation or support in addition to the fixation by adhesives, or by welding (such as laser welding, for example, with or without shrink tubing) or through the use of other adhesives or chemical and/or mechanical fixation expedients known in the art. For each end of coil 50, the wire of the coil 50 may circumscribe the tube for between about one to two full turns to enhance stability. The coils that circumscribe the tubing are adhered and reinforced by the shrink tubing that is shrunk outside of the coils, and against the tubing. Since there are inner and outer tubes, each coil end is attached in a similar manner to a respective one of the tubes.

FIG. 8H shows an end view of tubes 14 and 15 with coil 50 attached thereto, and showing the attachments 51,53 of coil 50 to tubes 14 and 15, respectively. Coil winding 52 is shown in an enlarged diameter configuration. Upon rotating tube 14 relative to tube 15 in a manner as described above, the diameter of coil winding 52 is reduced, as shown in FIG. 8I. For comparison purposes, attachment point 53 is shown in the same relative position in both FIGS. 8H and 8I, while attachment point 51 has been rotated about 270 degrees in FIG. 8I, relative to its position in FIG. 8H.

By laying out and attaching the edge 20e of the sheet material 20s to coil 50, tip 20 is formed with varying diameter functionality. Sheet material 20s is shown in planar form in FIG. 8J, prior to its attachment to coil 50 to form tip 20. FIG. 8K shows sheet material 20s attached to coil 50 to form tip 20. Note that the outside edge 20o is attached to the largest coil winding 52 of coil 50, which inside edge 20i is attached to an underlapping, coil winding 50. Attachment of edge 20e to coil 50 may be made using sutures, such as silk sutures, or other polymeric sutures known and used in the surgical arts. However, coil 50 (and particularly enlarged winding 52) may also function as an ablation element 12 in this example. Sutures, or the coil winding itself may be used to attach ablation element 12/coil 50 to the edge 20e. In order to be durable to heat, at least edge 20e of tip 20 should be made from high temperature plastic such as PEEK™ (polyether ether ketone resin,) or ULTEM® (amorphous thermoplastic polyetherimide), for example. If coil 50 itself does not make up the ablation element 12, ablation element 12 may be mounted on a distal end of a third tubing (not shown) that may be coaxially slid over the arrangement shown in FIGS. 8D to 8E to approximate the tissue radially surrounding edge 20e for ablation thereof. When coil 50 serves as ablation element 12 it is connected to a power source by extending one or more electrically connecting wires from coil 50 to the proximal end portion of instrument 10 in the same manner as described with regard to examples described above.

In the example shown, tip 20 is capable of varying outside diameters ranging from about 15 mm to about 20 mm. However, greater ranges of variation may be obtained, and also instruments having other ranges may be constructed. For example, an instrument 10 having variable diameters ranging from about 8 to 10 mm to about 15 mm, or ranging from about 20 mm to about 25 mm, or from about 15 mm to about 25 mm, or some other desirable range, may be constructed using the same principles and features described above.

A transparent and elastic seal may be provided over conical lens 20 to prevent blood flow between the overlapping ends 20o and 20i of tip 20. For example, a transparent, elastic membrane 21 may be mounted over the lens 20, thereby sealing the lens and preventing any fluid flow therethrough. At the same time, membrane 21 does not inhibit the relative rotation of the ends 20o and 20i with respect to one another, and expands or contracts to accommodate a change in size of the outside diameter of tip 20. Additionally, a sealing sleeve 54 (e.g., see FIG. 8L) may be provided over outer tubing 15, coil 50 and attaching to the proximal end of tip 20 to prevent blood/fluid flow into the coil and inside of instrument 10, thereby maintaining a clear field of view within the cavity defined by tip 20 for viewing through endoscope 16. Sleeve 54 is elastic so as to twist compliantly during relative rotations between tubes 14 and 15 and changes in the outside diameter of tip 20, thereby allowing the torsional movement of the coil 50 and tube 14 with respect to tube 15, while maintaining a fluid-proof seal. Elastic membrane 21 may be made from silicone, latex, or the like, for example. Twistable sealing sleeve 54 may be made from polyethylene, polytetrafluoroethylene, woven polyester, silicone, latex, combinations thereof, or the like, for example.

Further, a control mechanism may be provided between the proximal end portions of tubes 15 and 14 so as to maintain a desired tip diameter once the operator has adjusted the tip diameter as needed for a particular procedure. This eliminates the need to maintain torque between tubes 15 and 14 throughout the procedure, thereby freeing at least one hand of an operator for doing something else. It is also more accurate, as it may be difficult to maintain the outside diameter of tip 20 exactly the same throughout a procedure.

Tube 14 includes a torsion control grip 14H at a proximal end portion thereof that may be rotated to effect relative rotation between tubes 14 and 15. Torsion control grip may also act to prevent axial displacement of tube 14 distally with respect to tube 15. By grasping outer tube 15 to prevent its rotation and rotating torsion control grip 14H with another hand, relative rotation of the coil ends 51 and 53 can be effected, causing the diameter of coil winding 52 to increase or decrease by overlapping with adjacent coils of coil 50. FIG. 8M shows one example of control grip 14H in an unlocked position or configuration. A gear 14g is mounted to the proximal end of tube 14 and a swing arm 14s is mounted to outer tubing 15, such as by collar 14c or other alternative fixing arrangement. When swung out of the locking position, as shown in FIG. 8M, gear 14g (which may be ratcheted, or freely rotating) is allowed to rotate with respect to swing arm 14s to enlarge or reduce the size of tip 20 in a manner as described above. When the desired size of tip 20 has been achieved, swing arm 14s is rotated back towards gear 14g such that the tip of swing arm 14s, which may be in the form of a mating gear tooth, engages gear 14g between adjacent teeth of gear 14g, thereby preventing rotation of gear 14g with respect to swing arm 14s, see FIG. 8N.

FIG. 8O shows another example of control grip 14H in which detents 14d or depressions are formed in the proximal end of tubing 14. Arm 14a is fixed to the proximal end of tubing 15, such as by collar 14c or other means. Arm 14a includes a ball, bump or protrusion 14b that is configured to engage with the detents or depressions 14d. Thus, tubes 14 and 15 may be rotated with respect to one another to achieve the desired size of tip 20. When the desired size of tip 20 is achieved, protrusion 14b is maneuvered to engage with the nearest detent 14d, or the nearest detent in a particular direction (e.g., as in the case where the nearest detent in the enlarging direction is used, to ensure that the tip will not be undersized). Once engaged, tubes 14 and 15 are prevented from rotating with respect to one another under any biasing force that may be provided by coil 50, i.e., additional biasing force must be provided by the operator, such as by twisting tubing 15 with respect to tubing 14 in order to release protrusion 14b from engagement with detent 14d.

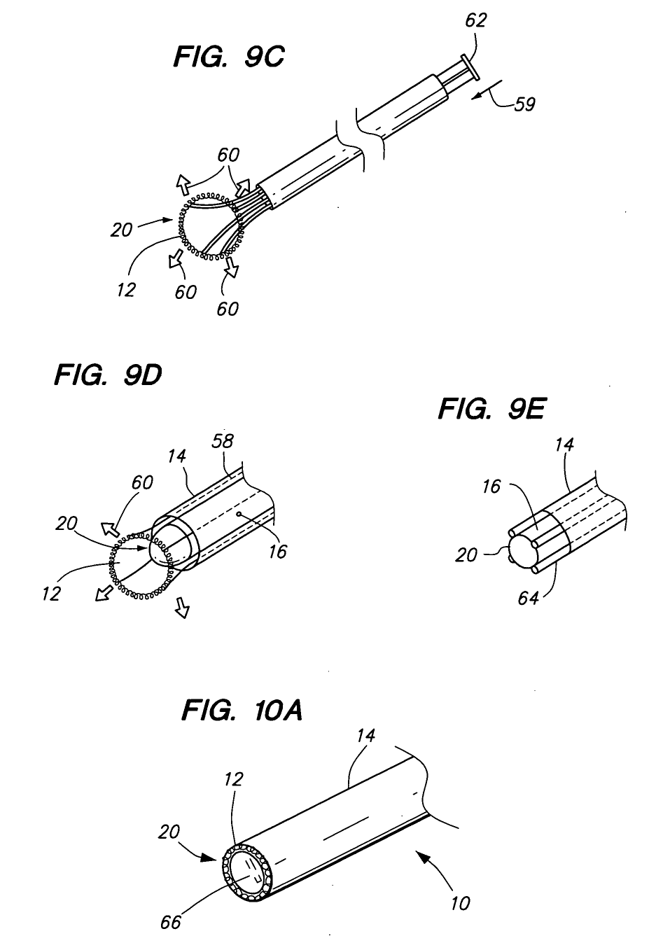

Another example of an ablation instrument 10 having a variable diameter tip 20 is illustrated in FIG. 9A. Variable diameter tip 20 facilitates delivery through a small opening (such as an atrial appendage, for example) during which time it is configured in a compressed or reduced diameter configuration. The diameter of tip 20 may then be increased to various larger diameter sizes which are useful for endocardial ablation around pulmonary vein ostia of different diameters, for example. In the example shown, tip 20 is an expandable ring, which may be formed, for example of an elastic spring coil, such as from any of the spring metals described above, or from a polymer having the appropriate spring characteristics for expanding the diameter thereof (without significant plastic deformation) from about 10 mm to about 40 mm or sub-ranges thereof, including from about 10 mm to about 30 mm, etc. Of course, other ranges may be designed using the same design principles, depending upon the particular surgical procedure to be performed, as well as any constraints that the delivery path of the instrument 10 may impose.

Control of the diameter of tip 20 is achieved through a plurality of rods 58 or stiff wires or other thin, elongated control members that are substantially rigid under compression but elastic in bending. As shown, tip 20 is controlled by four equally spaced control members 58, although more or fewer control members 58 may be connected to tip 20 to carry out the diameter control function. Control members 58 are each preformed into a curved configuration, as shown in FIG. 9B, so that when no biasing force is applied to control members 58, the distal ends thereof spread out to define the largest circumference/diameter. As control members 58 are slid proximally with respect to tube 14 and into tube 14 (in the direction of the arrow shown in FIG. 9B), the constraint of the tube 14 wall against the control members 58 puts a biasing force on control members 58 so that the circumference defined by the distal ends of control members 58 gradually decreases. When fully retracted into tube 14, control members are biased into substantially straight configurations, and the circumference defined by the distal ends of control members is about equal to or slight less than the circumference of tube 14. The bending (i.e., straightening) of control members 58 by tube 14 is carried out in elastic deformation only, so that when control members are again slid distally out of tube 14, they reassume the bent configuration that they assumed previously in their unbiased, preformed configuration. Intermediate positions between completely unbiased (maximum circumference) and straight (minimum circumference) configurations are continuously achievable by sliding control members with respect to tube 14, so that the diameter of tip 20 is continuously adjustable from the minimum possible to the maximum possible.

The pre-shaped, curved control members 58 may be formed from a shape memory material such as a nickel-titanium shape memory alloy or the like, or from any metallic rod or wire exhibiting the characteristics described above (rigidity in compression and elastic in bending). FIG. 9C illustrates distal movement of control members 58 (in the direction of arrow 59) with respect to tube 14 and the resulting expansion of tip 20 (in the directions of arrows 60). A control handle 62 may be provided proximally of the proximal end of tube 14 to facilitate equal translation/sliding of each control member 58 with respect to tube 14. In such case, control handle 62 is fixed to proximal end portions of each of control members 58, so that advancement or retraction of control handle 62 with respect to tube 14 advances or retracts control members 58 by equal distances.

Alternatively, pairs of control members 58 may be connected to separate handles, or each control member 58 may be driven independently. These configurations may be desirable if the operator wishes to expand distal tip to a shape that is non-circular, such as to an oval or oblong shape, by advancing control members 58 by different distances with respect to one another, or to establish an angled interface with distal tip 20. Typically, however, control members 58 are advanced and retracted by the same distances relative to tube 14.

Expandable ring 20 may also function as an ablation element in instrument 10, in which case, a source of power may be connected to ring 20/12 via one or more of control members 58 or via one or more separate wires through tube 14. Ring member/ablation element 20/12 need not be metallic or electrically conducting when the source of ablation is chemical or heating fluid, for example. As with the above embodiments, any of the ablation sources listed above may be applied through ablation element 12 in the example described with regard to FIGS. 9A-9C. Control members may be guided through separate ports, lumens or cannulae provided within tube 14, or may simply pass through tube 14 to abut against the inner wall of tube 14.

Further, the example described above with regard to FIGS. 9A-9C may also be used with an endoscope 16, much in the same manner as described with regard to above embodiments, and as illustrated in FIG. 9D. This configuration allows viewing of an ablation procedure with ablation effected by ablation element 12 and real time viewing through endoscope 16. Control members may be translated through the annular space provided between tube 14 and endoscope 16 as shown in FIG. 9D. Alternatively, separate tubing 64 may be provided between endoscope 16 and tube 14 to guide the movements of control members 58, as shown in FIG. 9E, where tube 14 is shown partially cut away. Tubes 64 are particularly useful when a dome-shaped lens 20 is provided for use with endoscope 16, as described previously, as this creates an annular space for control of the endoscope shaft 16 for changing depth of view.

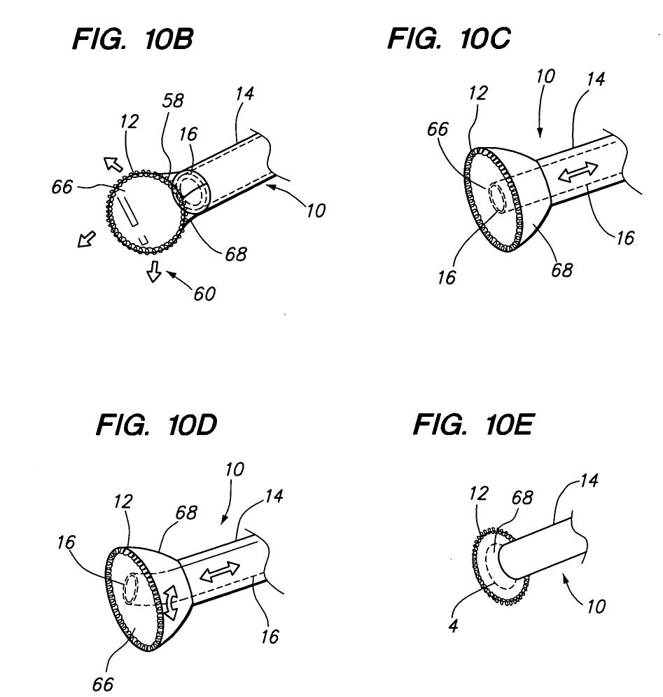

FIG. 10A illustrates another ablation instrument 10 with a varying diameter tip portion. Instrument 10 is similar to the instruments described above with regard to FIGS. 9A-9E in that it includes an expandable ring, which may be an expandable ablation element 12. However, instrument 10 of FIG. 10A includes an expandable transparent diaphragm 66 spanning expandable ring 12 that may function as a lens for endoscope 16, thereby obviating the need for a spherical or other lens mounted on the distal end of endoscope 16 (such as in the configuration in FIGS. 9D-9E, for example). Expandable diaphragm 66 may be made of silicone or latex, or the like, for example, and seals with expandable ring 12 to prevent blood flow through ring 12. FIG. 10A shows ring 12 and diaphragm 66 in the smallest diameter configuration and FIG. 10B shows ring 12 and diaphragm 66 in an expanded configuration having a substantially larger diameter.

The space between the distal end of tube 14 and expandable ring 12 is joined and surrounded by covering or seal 68 to seal off the cavity defined by ring 12, control members 58 and the distal end of tube 14, to provide a clear and clean cavity for viewing procedures via endoscope 16. Seal/covering 68 is elastic in both elongation (unless it is formed to be bellows-like) and radial directions to accommodate changes in distances as the control members expand out, and should not be so stiff as to prevent control members from expanding. Seal/covering 68 may be made from silicone or latex (elastic) or woven polyester (cloth-like) or combinations thereof, for example. It may be folded or crumpled up (or bellows-like) to provide capacitance for linear expansion thereof. Thus, seal collar 68 prevents blood inflow into the cavity.

Elastic diaphragm 66 may eliminate the need to have a dome-shaped or other lens distally mounted in front of endoscope 16. The camera for endoscope 16 may need to have enhanced focusing capability for a configuration as shown in FIG. 10B when endoscope 16 is not translatable distally from the distal end of tube 14. For example, a Stryker's endoscope (Stryker Communications, www.strykercorp.com) or similarly performing endoscope may be employed in order to have sufficient focusing capability as lens/elastic diaphragm 66 moves away from endoscope 16 at the same time that it expands.

Alternatively, endoscope 16 may be configured to translate axially, distally of the distal end of tube 14, as shown in FIG. 10C. With this capability, endoscope may be distally translated proportionately to the distal advancement of diaphragm 66, relative to tube 14 as control members 58 are distally advanced to expand ring 12, thereby greatly lessening the focusing requirements of the endoscope camera, since focusing can be accommodated by translation of endoscope 16. It may be further advantageous to provide a flexible or articulating endoscope 16, as shown in FIG. 10D to allow panning of the view, particularly when ring 12 and diaphragm 66 are expanded to or near the maximum end of the expansion range, although articulation may also be performed (although needed less) for smaller diameter configurations of ring 12.

FIG. 10E illustrates an ablation instrument 10 of the type described in FIGS. 10A-10D, in position to perform an ablation. After insertion through the atrial appendage with ring 12 in a contracted configuration (FIG. 10A), endoscope 16 is used by the operator to view the endocardial wall of the atrium. Upon locating a pulmonary vein ostium, the operator continues viewing through endoscope 16 to provide visual feedback for aligning/centering instrument 10 with the pulmonary vein ostium that the operator intends to form a lesion around. Once centered, or in the vicinity thereof, control members 58 are advanced distally with respect to tube 14, while viewing the progress of the expansion of ablation element 12 through endoscope 16. When the operator has visually determined that ablation element 12 is of a sufficient size to surround the ostium and provide a border of endocardial (atrial) tissue, between ablation element 12 and the perimeter of the ostium, element 12 is centered (if not already centered) again while providing visual feedback through endoscope 16. Once centered, ablation element 12 is approximated to the endocardial tissue surrounding the ostium and ablation energy (of whatever form) is then applied through ablation element 12 to begin the ablation process. Continued viewing through endoscope 16 may provide visual feedback as to the progression of the lesion formation, such as by viewing tissue blanching as described above. The procedure may be interrupted to view the lesion after removing ablation element 12 and then ablation element and ablation energy can be reapplied as necessary, or it may be possible to continue the procedure all the way though until completion of the lesion is confirmed by visualization and/or other forms of monitoring.

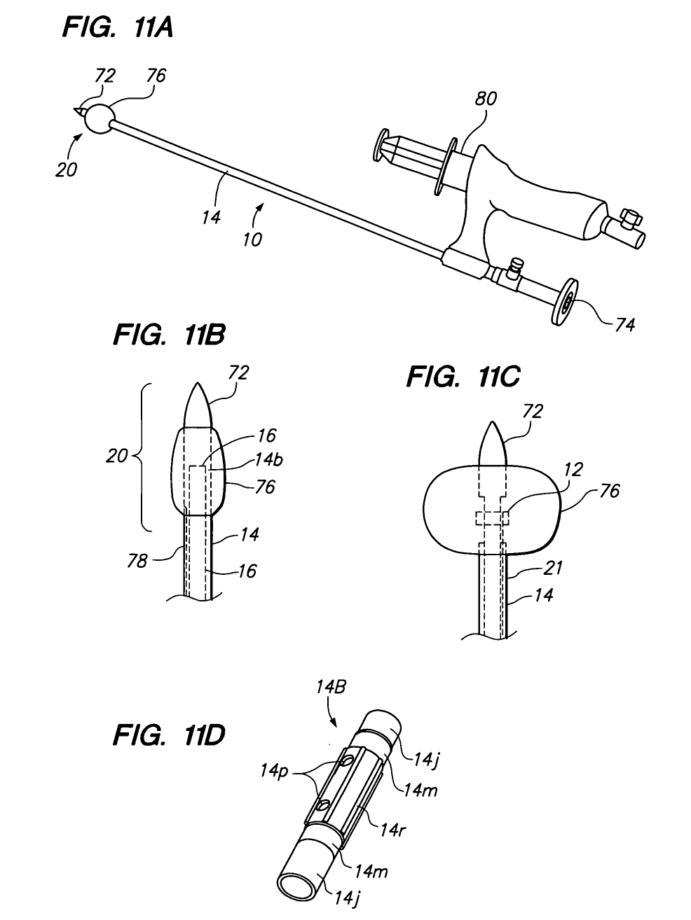

FIG. 11A shows a perspective view of an ablation instrument 10 configured for applying ultrasonic energy to perform ablation. Instrument 10 includes rigid tube 14 (which may alternatively be malleable, as discussed above with regard to previous examples, but must maintain sufficient rigidity after bending, such as by hand, for example, so that it does not bend during use) that houses endoscope 16 in a manner similar to that described above. The tip portion 20 of instrument 10 includes a distally mounted, transparent (optically clear) distal tip 72 mounted at the end distal end of tube 14. In the example shown in FIG. 11A, endoscope 16 is arranged to view only the tip 72. However, endoscope 16 may be slidably mounted with respect to tube 14, tip 72 and balloon portion 76 of tip 20, in a manner as described above and as illustrated in FIGS. 11B-11C, so that the axial position of the endoscope 16 can be varied to view tip 72 or tip 72 and balloon 76.

In embodiments where the endoscope 16 is axially slidable, after inflating balloon portion 20, endoscope 16 is slid distally, so that the tip of endoscope 16 enters a space defined by the inflated balloon 76. In this position, the entire ostium border of a pulmonary vein ostium can be viewed through balloon portion 76, as tip 72 is inserted towards and into the ostium. Tip 72 is attached to tube 14 and is not expandable. When balloon 76 approximates the ostium, the ostium is clearly visible by endoscope 16, viewing through the wall of balloon 76. Tip 72 will be visualized in red, indicating that the device is properly centered in the ostium, since blood exists all around tip 72. Tip 72 may be made from glass, polycarbonate, PET, polyester, high durometer silicone, high durometer polyurethane, or the like, and may have a diameter of about 5 to about 9 mm, typically about 7 mm.

Thus, the distal end of endoscope 16 is positioned to enable viewing of the ostium from the proximal end of instrument 10 through window 74. Through the tip 72, only a portion of the ostium can be visualized at any one time. However, by axially retracting (proximally) the endoscope 16 relative to tip 72 for viewing through inflated balloon 76, the entire ostium can be viewed.

A balloon mount segment 14B interconnects the remainder of tube 14 with tip 72. Balloon mount segment (see FIG. 11D) may be made of plastic, typically clear plastic, such as from polycarbonate, SAN (styrene acrylinitrile), ABS (acrylonitrile-butadiene-styrene), acrylic, PET (polyethylene terephthalate), polyester, or other polymeric resin, and may be made from the same or different material as tube 14. Segment 14B may include mounting features, such as ribs 14r to which ablation element 12 may be mounted, such as by gluing (adhesives), mechanical fixation (friction fit or other mechanical fixation) or a combination thereof. Openings, holes or ports 14p are provided through segment 14B through which pressurized fluid may be delivered to inflate balloon 76 when mounted over segment 14B in a manner as described hereafter. Mounting surfaces 14m are provided to which proximal and distal ends of balloon 76 may be fixed, respectively, to create fluid/air tight seals between the balloon 76 and segment 14B. Such fixation may be performed by adhesives, sutures or a combination of the two, or, alternatively, by other mechanical fixation techniques, together with adhesives. A lap joint or other surface 14j is provided at proximal and distal ends of segment 14B for fixation to tube 14 and tip 72, respectively. Such fixation is typically performed using adhesives, but may additionally or alternatively be performed with the use of friction fitting, heat welding, laser welding, or other fixation techniques.

Ablation element 12 (such as a piezo-electric crystal) is cylindrical and has an inside diameter large enough to accommodate endoscope 16, and balloon 76 is mounted over the outside of ablation element 12. Ablation element 12 is mounted to balloon mount segment 14B as described above, and then balloon 76 is mounted over ablation element 12 and sealed at proximal and distal ends as described above. When ablation element 12 includes a piezo crystal, ablation element is typically mounted by interference fit or flexible adhesive (such as RTV (room temperature vulcanization) or silastic adhesive). Typically, balloon 76 is glued and optionally overtied onto balloon mount grooves 14m. Thus, balloon mount segment 14B is provided in the annular space between ablation element 12 and endoscope 16, and balloon 76 is axially mounted over balloon mount segment 14B proximally adjacent tip 72. Balloon 76 may be a high pressure, semi-rigid inflatable toroidal balloon made from a material such as polyethylene, polyvinyl chloride, polyethylene terepthalate, or the like, or may be made from an elastic material such as polyurethane, silicone or latex, or the like, for example, wherein, when an elastic material is used, balloon 76 is inflated to the extent that an elastic limit is reached so that balloon 76 becomes semi-rigid during use. In a deflated state, as shown in FIG. 11B, balloon 76 closely approximates the outside diameter of tube 14 to facilitate insertion of tip portion 20 through a small opening. An inflation lumen 78 is provided in tube 14 to fluidly connect balloon 76 with a source of fluid 80. After placement of tip portion 20 into the surgical target area (e.g., after passing tip 72 and deflated balloon 76 through the atrial appendage), liquid is supplied under pressure to balloon, such as by syringe 80 or other pressurized liquid driver, for example, to inflate and pressurize balloon 76 with fluid, forcing it to assume the expanded configuration shown in FIG. 11C. A stopcock or other shutoff device may be provided in the line connecting the pressurized fluid source 80 with balloon 76 which can be shut off to maintain expanded balloon 76 under fluid pressure, in a manner similar to that described above with regard to the example of FIG. 6H. The size (outside diameter) of balloon 76 is adjustable by the volume of fluid (e.g., saline) that is pumped into it under pressure. Balloon 76 is semi-rigid, having sufficient rigidity so that the wall of balloon 76 will not conform to the shape of the ostium upon approximation therewith, unless the operator applies excessive force. The expanded diameter of balloon 76 is larger than the inside diameter of the ostium that tip 72 approximates, thereby making it physically impossible for balloon 76 to enter the ostium, and ensuring that energy delivered through balloon 76 does not enter the ostium, so that lesions are created in the endocardial wall (of the atrium) surrounding the ostium and not in the ostium.

Ablation element 12 is located concentrically inside balloon 76 and concentrically outside endoscope 16 as noted above. In this example ablation element may be an ultrasonic transducer or an array of ultrasonic transducers that are connected to a source of energy located proximally outside of device 10, via one or more electrically conducting connecting wires 21. Ablation element 12, when energized, transmits energy from the ultrasonic transducer(s) through the fluid in expanded balloon 76 to any tissue contacting balloon 76.

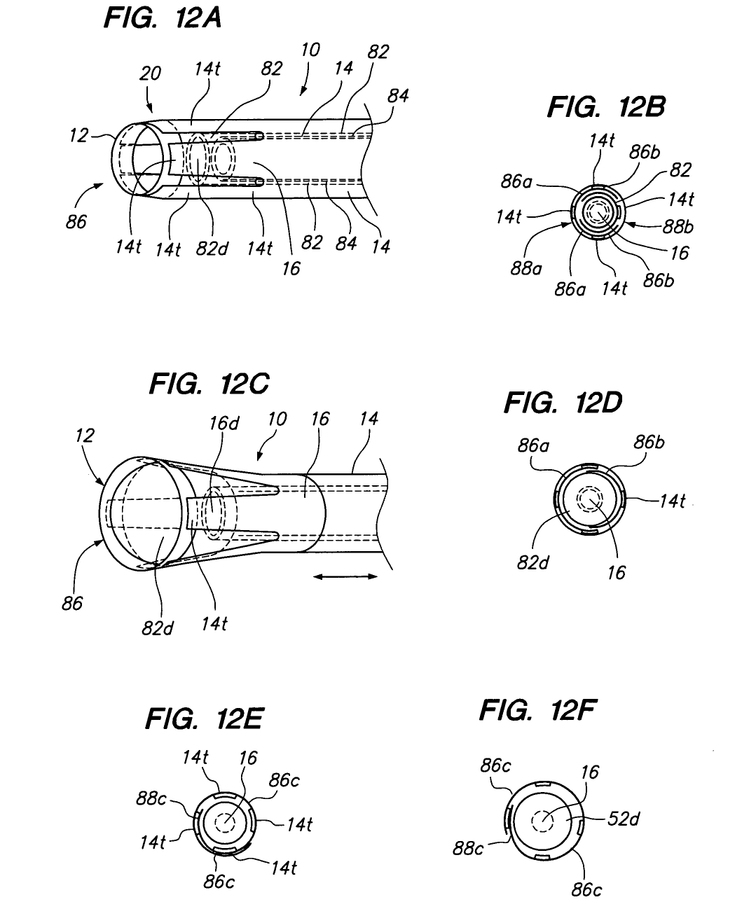

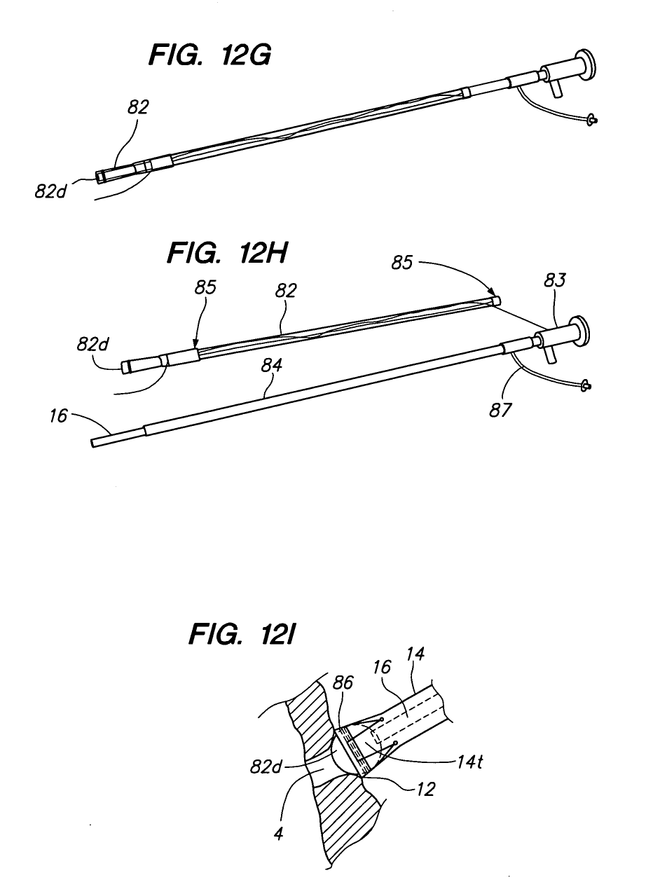

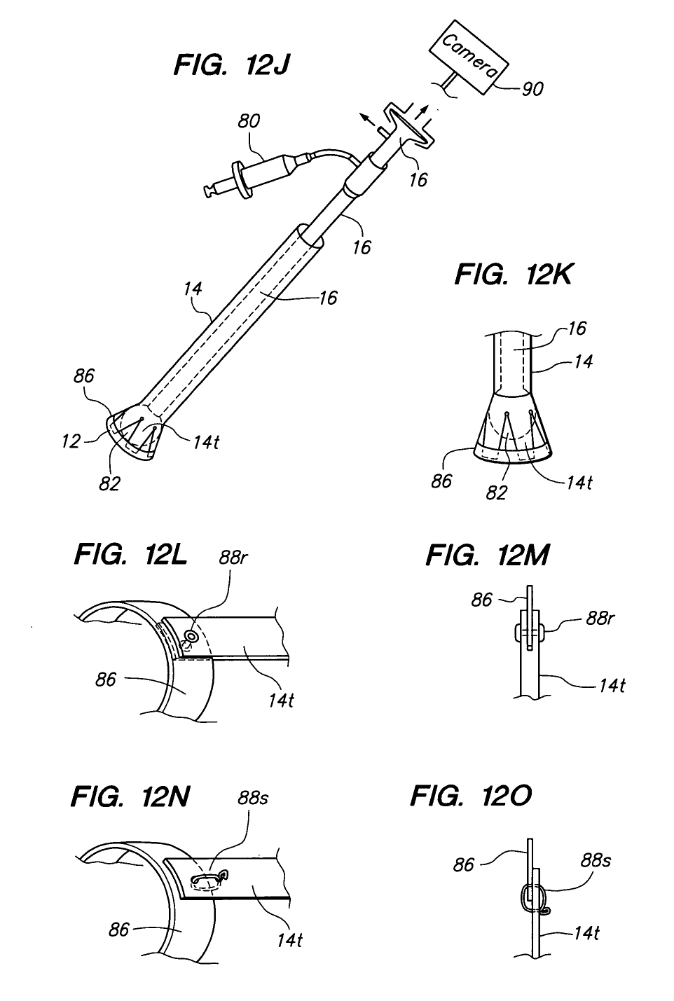

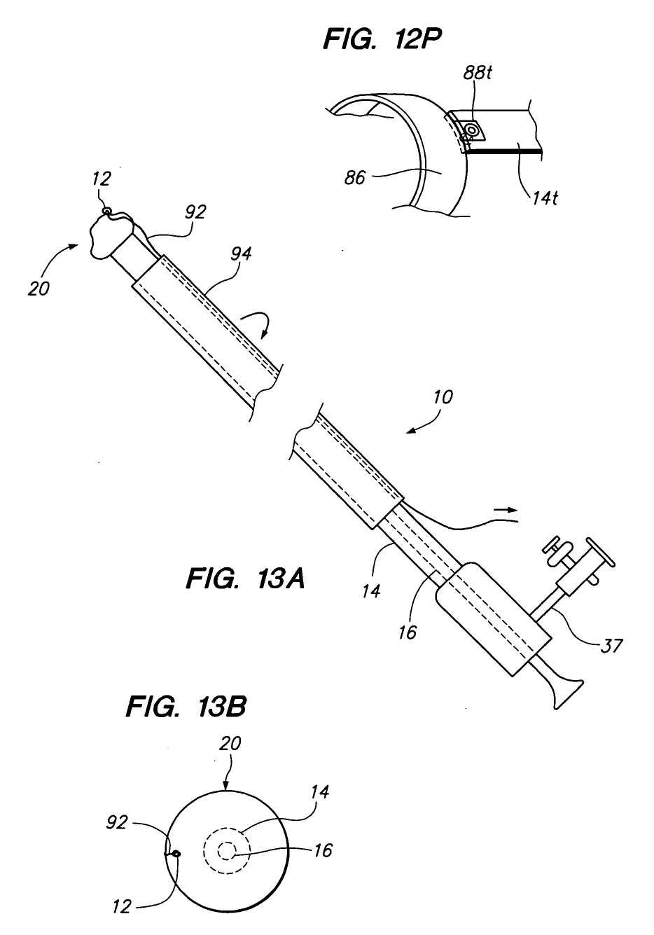

Referring now to FIG. 12A, another example of an ablation instrument having a tip portion that is adjustable in size is shown. A rigid outer tube 14 (which may alternatively be malleable, as described above) is provided through which endoscope 16 is axially received, similar to embodiments described earlier. In this example however, the distal end portion of tube 14 is formed as split tubing 14t that is flexible to the extent that it is expandable by force applied to it during expansion of expandable member/lens 82d Expandable member 82d may be formed as an elastic balloon member (e.g., silicone or latex, or the like) having a substantially flat distal surface that closes the distal end of tubing 82. Endoscope 16 is axially received within tubing 84 that is, in turn, axially received within tubing 82. Tubing 82 is provided with passive ring seals 85 (FIGS. 12G-12H) in locations to form a liquid tight seal with tube 84 even when tube 82 and tube 84 are slid axially with respect to one another. Endoscope 16 is axially fixed with respect to inner tubing 84, and an annular space is formed between the inner wall of inner tubing 84 and the outer wall of endoscope 16. the annular space is closed off at the proximal end by the ocular/connector for the camera of the endoscope. An inflation port 87 is provided through tubing 82 to allow an inflation fluid (e.g., saline, or the like) to be injected under pressure to be delivered through the annular space/lumen 84 to expandable member 82d to drive the expansion of the same. The distal proximal ring seal 85 seals the space between tube 82 and tube 84 to prevent backflow of the pressurized fluid proximally thereof.

An expandable ring 86 is mounted over the distal ends of the split portions 14t and is configured to expand in perimeter/diameter when driven to such a configuration by the expanding split tube portions 14t as they are in turn forced to expand by the expanding balloon 82. FIG. 12B shows an end view of instrument 10 of FIG. 12A in a smallest diameter configuration, where it can be seen that a portion of the expandable ring 86a substantially overlaps another portion 86b, to ensure continuity of the distal ring edge even in the most expanded configuration of expandable ring 86. Expandable ring is fixed or mounted to split tube portions 14t, such as at 88a and 88b, for example, although additional points of attachment may be made such as ninety degree angles to the two locations described, for example. Having more than two attachments points/locations may avoid the coiling/wrapping of a longer ablation ring, that is, a shorter length of ablation ring may be able to be used, thereby reducing friction between the sliding coils. More than two attachment locations may also provide for a more uniform opening as driven by the fixed attachment points rather than simply depending upon the coiling/uncoiling movement to conform to the circular shape of the expanding tubing. The expandable ring may be fixed to the split tubing members by adhesive, suturing by drilling a hole through the coil and the tubing member and tying a suture through the aligned holes, a mechanical locking arrangement such as a slit on each one of the attaching split tube members and a mating tab for each of the slits on the coil, or some combination of the foregoing, for example.

FIGS. 12L and 12M show a partial perspective view and a partial side view, respectively, of a device showing fixation of ring 86 to split tubing 14t via rivet 88r. A slot is made in the distal end of each split portion of tubing 14t and ring 86 is slidably received therein. A through hole is then made through each split tubing member 14t and portion of ring 86 wherein fixation is to be made, and a rivet, pin, bolt and nut, or the like 88r is secured therethrough. FIGS. 12N and 12O show a partial perspective view and a partial side view, respectively, of a device showing fixation of ring 86 to split tubing 14t via suture 88s. In this arrangement, a through hole is made in ring 86 where fixation by suture is to be accomplished, and two holes are made in the adjacent split tubing 14t. A suture is then passed trough all holes and knotted as shown, to fix a portion of ring 86 to a split tubing portion. FIG. 12P shows a partial perspective view of a device showing fixation of ring 86 to split tubing 14t via a tab and slot arrangement. In this arrangement, a slot is made in the distal end of each split portion of tubing 14t, similar to that described with regard to FIGS. 12L and 12M above. In this example, however, ring 86 is provided with tabs 88t extending proximally therefrom, which are slidably received in the slots of split tubing 14t. The slotted portions of the split tubing members may contain detents or other mechanical fixation members extending inwardly to engage a dimple, through hole or other mating mechanical fixation member in tab 88t. Of course the fixation members may also be reversed, with one or more male mating members extending from tab 88t and a female mating member(s) on the slotted portions. In any case, the elasticity of the slotted portions of split tubing members 14t allow slight deflection thereof when tabs 88t are slid therebetween after which the slotted portions snap back into place to complete the fixation of the ring 86.

Upon inflating expandable member 82, the elastic balloon member both lengthens and expands in diameter, as illustrated in FIG. 12C, thereby deflecting split tube portions 14t to a larger diameter configuration which enlarges the perimeter of expandable ring 86. When expanding, the split ring portions 86a,86b slide against each other to enlarge the perimeter/diameter as shown in FIG. 12D. Endoscope 16 may be slid axially with respect to tube 14 (in the directions of the arrows shown in FIG. 12C) to vary the focusing capabilities/field of view through endoscope 16 and camera if attached thereto. For example, endoscope 16 may be moved distally with respect to tube 82 to position the distal end 16d thereof within the expandable member 82d to provide better visualization of expandable ring 86.

In one example of use, the distal end portion (including the distal tip configuration described above with regard to FIGS. 12A-12B) is inserted through the atrial appendage with expandable member 82, split tube portions 14t and expandable ring 86 all in their contracted, smallest diameter configurations. Endoscope 16 may be used by the operator to view the insertion through the atrial appendage and, after the insertion has been accomplished, to view the endocardial wall of the atrium. Upon locating a pulmonary vein ostium, the operator continues viewing through endoscope 16 to provide visual feedback for aligning/centering instrument 10 with the pulmonary vein ostium that the operator intends to form a lesion around. Once centered, or in the vicinity thereof (or even before a centering procedure has begun, as long as distal tip portion is inside the atrium), balloon 82 is filled with pressurized fluid to expand it, slit tube portions 14t and expandable ring 86 to enlarged diameter configurations, such as shown in FIGS. 12C and 12D.

The progress of the expansion may be continuously or intermittently viewed through endoscope 16. Once expanded or during expansion, the operator may move the endoscope distally with respect to tube 14 to place the distal end of endoscope 16 closer to the distal end of instrument 10, including to positions within the expandable member 82. When the operator has visually determined that ablation element 12 (mounted on the distal end of ring 86) is of a sufficient size to surround the ostium and provide a border of endocardial (atrial) tissue, between ablation element 12 and the perimeter of the ostium, ring 86/element 12 is centered (if not already centered) while providing visual feedback through endoscope 16. Once centered, ablation element 12 is approximated to the endocardial tissue (either pressed in contact against, or positioned at a desired distance therefrom for forming a lesion, depending upon the energy source used for ablation) surrounding the ostium and ablation energy (of whatever form) is then applied through ablation element 12 to begin the ablation process. Continued viewing through endoscope 16 may provide visual feedback as to the progression of the lesion formation, such as by viewing tissue blanching as described above. The procedure may be interrupted to view the lesion after removing ablation element 12 and then ablation element and ablation energy can be reapplied as necessary, or it may be possible to continue the procedure all the way though until completion of the lesion is confirmed by visualization and/or other forms of monitoring.

One or more connecting wires or conduits are provided to connect expandable ring and particularly ablation element 12 to a source of ablation energy, through (or inside of) tube 14, where the ablation energy source is located proximally, outside of instrument 10 (not shown). When an electric current is provided to ablation element 12, such as when ablation element 12 applies Rf energy, microwave energy or resistive heating for example, expandable ring 86 may be metallic and split tubing 14t must be able to withstand heat generated by ablation element 12 and conducted through expandable ring 86. In these arrangements, split tubing 14t may be made of heat-resistant plastic such as ULTEM® (amorphous thermoplastic polyetherimide), or polyether ether ketone, or similar material. Such materials are used in a thickness so that they are readily deformed by the expansion of expanding member 82 and further provide both heat and electrical insulation to the surrounding environment. Expandable member 82 may be made from a transparent biocompatible elastomer such as silicone, latex rubber, or the like to provide compliance for variation in size (expansion and contraction) upon filling it with pressurized fluid (such as saline, for example) and removing fluid therefrom, with restriction in its shape provided by its surrounding borders. Thus, split tubing 14t and expandable ring 86 allows axial elongation of expandable member without restraint, and radial expansion is greater at the distal end of instrument 10 (distal end of expandable member 82) than at the proximal end portion of expandable member 82 where split tubing members 14t are relatively wider (optionally thicker) and stiffer, being nearer the unsplit tube 14.

In arrangements where electricity is supplied to ablation element 12 to perform ablation, expandable ring 86 may be formed of a metal having good electrical conduction capabilities, and has a natural characteristic to form a smaller diameter ring when under no biasing force (i.e., the contracted configuration shown in FIG. 12B), although even if the expandable ring does not have this natural characteristic, it should be compliant enough to assume the contracted configuration under slight biasing force by the natural tendency of split tube members 14t toward the contracted configuration. For example, expandable ring may be made of spring steel that has been pre-coiled to assume the configuration shown in FIG. 12B, or alternatively, a nickel-titanium alloy although these are not as good conductors of electricity as steel. In embodiments where the expandable ring does not need to conduct electricity, the expandable ring may be made of plastic, such as plastic shim stock (polycarbonate sheet) or other polymer with similar performance as would be apparent to those of ordinary skill in the art.

An alternative to the split ring portions 86a,86b may be employed in the form of a coiled ring 86c, as shown in the end views of FIGS. 12E and 12F. In this arrangement, coiled ring 86c is formed of a single continuous overlapping coil and is fixed to only one split tubing member 14t at 88c, for example. Split tube members 14t abut coiled ring 86c from radially inside positions, so that both the expansion of expandable member 82 and the expanding split tube members 14t provide radially expanding forces to coiled ring 86c causing the coils of ring 86c to slide with respect to one another (and over split tube members 14t, except for the one that is fixed to ring 86c). FIG. 12F shows expandable ring member 86c having been expanded in the manner described.

Further optionally, expandable member 82 may be further inflated to expand the substantially flat face 82d into a convex surface extending distally beyond ablation element 12 as shown in FIG. 12G. The convexly expanded distal surface 82d of expandable member 82 has a diameter that is larger than the inside diameter of ostium 4, thereby making it impossible to insert the expandable member 82 into ostium 4 and also ensuring that the diameter of expandable ring 86 and ablation element 12 are greater than the inside diameter of ostium 4 by a margin that ensures that only endocardial tissue will be ablated, so that the lesion formed does not contact the ostium. As noted earlier, endoscope 16 may be axially slid distally with respect to tube 14 to improve viewing of the expandable ring 86/ablation element 12 and to visually ensure that a margin of endocardial tissue lies between ablation element 12 and ostium 4 before commencing the ablation.

FIG. 12I illustrates a partial perspective view of an ablation instrument 10 of the type described above with regard to FIGS. 12A-12H. In this example, a 7 mm endoscope is provided within a plastic (e.g., ULTEM®, ABS, polycarbonate, etc.) tube 14 having distal split tube portions 14t. The plastic material chosen for tube 14 is chosen for its heat resistance properties and its ability to flex and not plastically deform within the range of motion during expansion and contraction motions A syringe 80 was used to supply saline under pressure to inflate/expand expandable member 82. The proximal end of endoscope 16 was connected to a camera/monitor 90. Expandable ring 86 is of the split ring variety that was discussed above. In this example, expandable member 82 is sealed directly over the distal end of endoscope 16, so that endoscope 16 cannot be moved axially with respect to expandable member 82. However, endoscope 16, together with expandable member 82 can be moved axially with respect to tube 14.

In this example, after insertion through the atrial appendage and expansion of expandable member 82, expandable member was slightly deflated and endoscope 16, together with expandable member 82, were retracted slightly (less than 5 mm) with respect to tube 14 in order to provide a better view of expandable ring 86, as shown in FIG. 12K.

FIG. 13A is a perspective illustration of another example of an ablation instrument 10. In this example an elastic tip member 20 is sealed over the distal end of tube 14 and tube 14 houses endoscope 16, as in previous examples. Ablation element 12 in this example, is a single element, such as a single electrode, or other single element that does not circumscribe tip 20 (see also the end view of FIG. 13B). In the example shown, ablation element 12 is of a type that is supplied by an electric power source and one or more electrically conductive wires A very thin plastic tubing or sheath may be provided over wire(s) 92 and around a portion of tube 14, to prevent wire(s) 92 from straying or becoming separated from instrument 10, while at the same time allowing wire(s) 92 to slide as more wire length will be taken up by the expansion of tip 20 discussed below.

Tip 20 may be formed of a transparent elastomer such as silicone or latex rubber, or the like, and is expandable by supplying fluid (such as saline, for example) under pressure through port 37, in a manner described previously. Upon expansion, tip 20 takes on a convex shape and has a diameter that is greater than an inside diameter of an ostium around which an ablation is to be performed. Endoscope 16 is available for viewing the ostium and the amount of expansion of tip 20 as it is inflated to ensure that ablation element lies outside of the ostium when instrument 10 is centered on the ostium, and that a margin of atrial tissue exists between ablation element 12 and the periphery of ostium 4. FIG. 13B shows an end view of tip 20 in an expanded configuration.

When tip 20 has been expanded sufficiently to meet the conditions described above, as confirmed by visualization through endoscope 16, instrument 10 is advanced distally to contact ablation element 12 against the endocardial tissue outlying ostium 4. Energy is then supplied to ablation element 12 to begin the ablation. The ablation may be visually observed via endoscope 16 as the ablation proceeds. The operator gradually rotates instrument 10 (with expanded tip 20 cannulated in ostium 4) to circumscribe the ostium with ablation element 12 thereby forming a circumferential lesion in the atrial tissue surrounding the ostium 4.

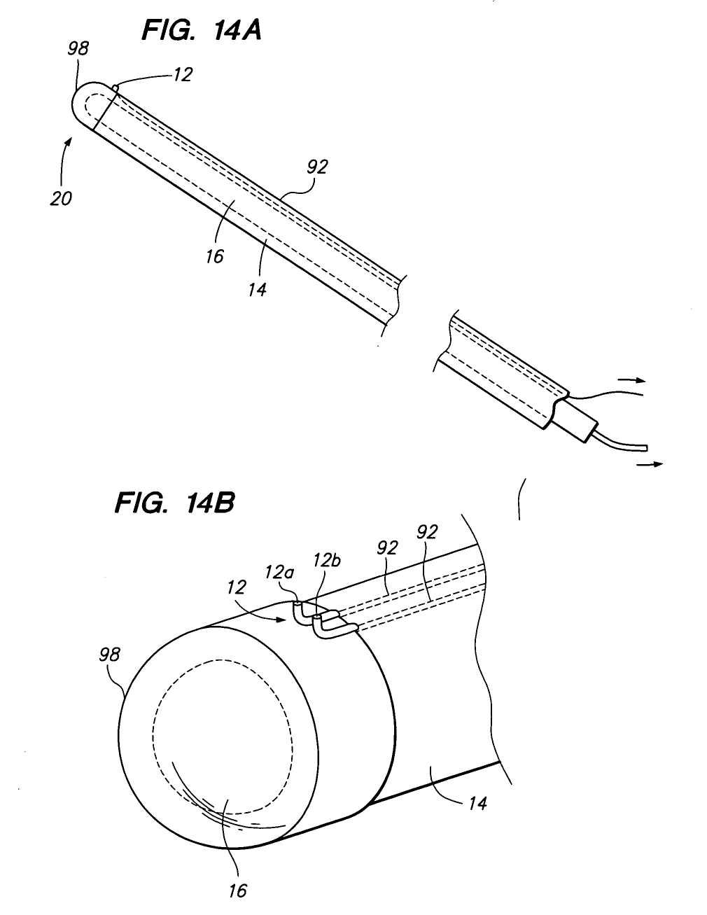

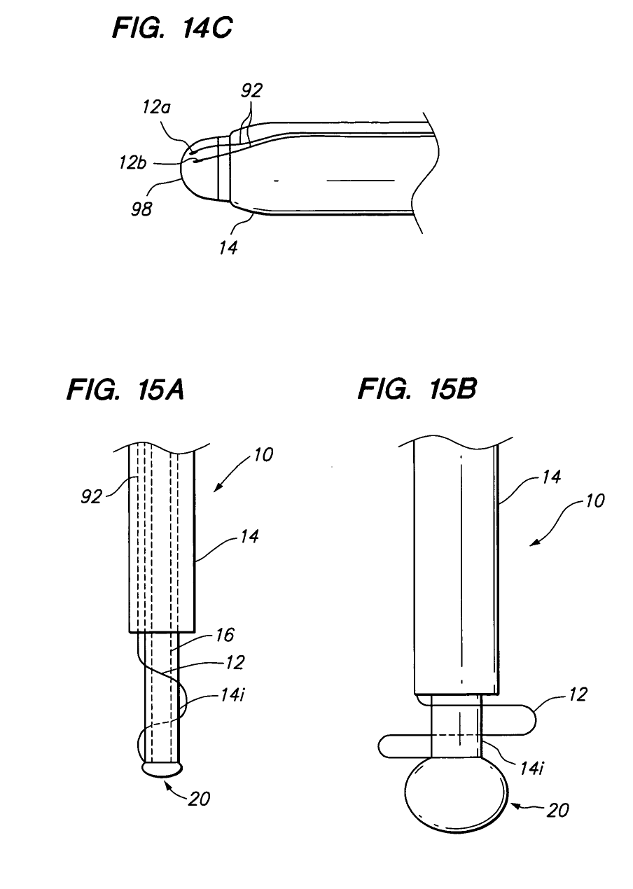

FIG. 14A is an example of another ablation instrument 10 which is configured to drag the tip portion 20 in order to form a lesion via ablation. In this example, endoscope 16 (such as an endoscope of the type noted above with regard to FIG. 3A, for example) is axially provided within rigid (or malleable) outer tube 14. The tip portion 20 of instrument 10 includes a transparent blunt-curved tip, such as rigid spherical tip 98 that permits tip portion 20 to be dragged over the endocardial (or other) tissue while at the same time viewing the tissue through endoscope 16, without damaging the tissue. Blunt tip 20 allows rapid identification of the cardiac anatomy as the device is dragged along the structures inside the heart for visual identification of the location(s) where it is desired to perform ablation(s). Ablation element 12 is mounted on the periphery of blunt tip 20 so as to be viewed by endoscope 16 and to approximate the tissue that tip 20 is dragged over.

In the example shown, spherical tip 98 was machined from polycarbonate and vapor polished. Ablation element 12 was made from a pair of electrodes 12a,12b (see FIG. 14B) for performing bipolar Rf ablation, although other types of ablation elements/ablation energy sources may be substituted, and other materials, configurations of tip 20 may be used alternatively. KYNAR® (polyvinylidene fluoride) coated wires 92 were implanted in the tip 98 with the electrodes 12a,12b extending slightly out from the surface (or flush therewith, but with contacts exposed, see FIG. 14C) for application of bipolar energy to the tissue to be abated. For an instrument that uses an endoscope having a five millimeter outside diameter, the outside diameter of tip 98 may be about ten millimeters, and the outside diameter of tube 14 may be about 7.5 millimeters. The endoscope may be translated with respect to tube 14, if desired.

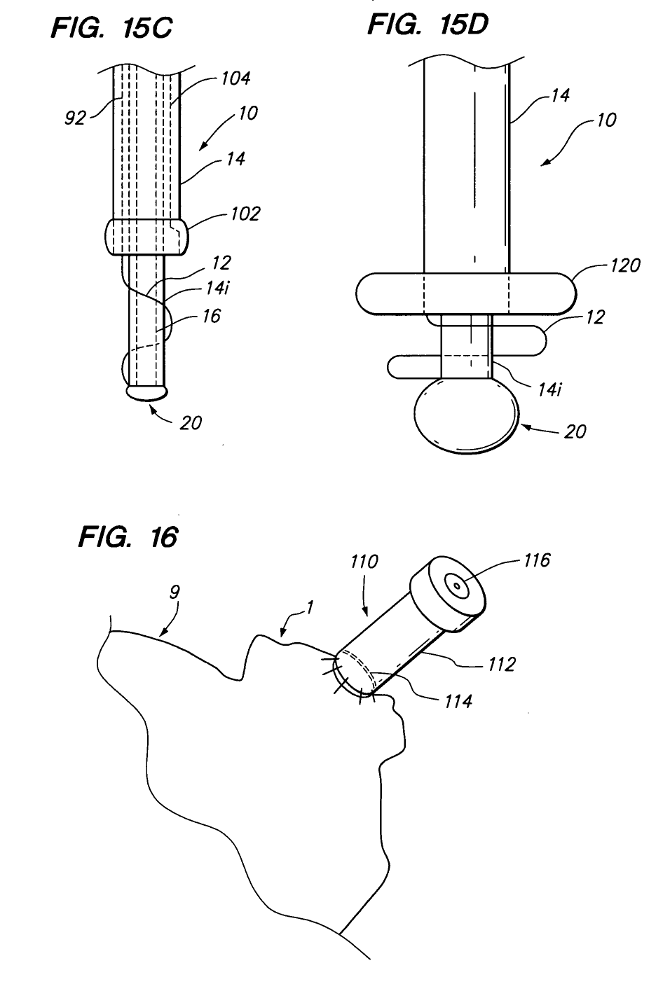

FIG. 15A is a partial view of a telescoping ablation instrument 10 that is adapted to perform endocardial ablation or other closed spaced ablation procedures via delivery through a small opening in a patient. Instrument 10 includes outer tubing 14 and inner tubing 14i in which endoscope 16 is axially, concentrically positioned. Inner tubing 14i telescopes with respect to outer tubing 14 (i.e., translates with respect thereto). Further, endoscope 16 is axially translatable within inner tubing 14i, with respect to inner tubing 14i. Transparent tip 20 is provided over the distal end of instrument 10 for viewing therethrough using endoscope 16. Tip 20 is blunt, and may be hemispherical or other flat or curvilinear blunt shape to prevent damage to tissues upon contact therewith or sliding thereover.

Ablation element 12 is a spiral wire or other elastic fiber, which is also electrically conducting when ablation is to be performed using Rf energy, microwave energy or resistive heating for example. Spiral ablation element 12 is fixed with respect to the distal end portion of outer tube 14 at one end and with respect to the distal end portion of inner tubing 14i at the other (distal) end. As noted above, outer tube 14 and inner tubing 14i may telescope or slide with respect to one another. The instrument is shown in the retracted or “telescoped out” position in FIG. 15A.

In the telescoped out position, the relative positions of the distal ends of tube 14 and inner tubing 14i elongate ablation element 12 causing it to assume a configuration of minimal circumference/outside diameter. This configuration is optimal for inserting the distal end portions of inner tubing 14i and tube 14 through a small opening in a patient for use in a closed surgical operating site. Once distal tip 20 and the distal end portions of inner tube 14i and tube 14 have been inserted beyond the small opening (such as an opening in an atrial appendage, for example), outer tube 14 may be “telescoped in”, i.e., slid axially in a distal direction with respect to inner tubing 14i, as shown in FIG. 15B.

By telescoping in tube 14, the distal end of tube 14 is moved substantially closer to the distal end of inner tube 14i, thereby significantly shortening the distance between the fixed proximal and distal ends of ablation element 12. This forces ablation element 12 to assume a much larger diameter/outside diameter, as shown in FIG. 15B. For example, in the telescoped out position (FIG. 15A) the spiral formed by ablation element 12 may have an outside diameter of about 10 mm, while in the fully telescoped in position (FIG. 15B), the spiral formed by ablation element 12 may have an outside diameter of about 25 mm. Intermediate telescoping positions of tube 14 with respect to endoscope 16 may also be established, so that the outside diameter of the spiral formed by ablation element may be continuously varied between the smallest diameter (FIG. 15A) and the largest diameter (FIG. 15B). Such ability to establish intermediate outside diameter sizes of ablation element 12 is very useful for performing lesions of various diameters, such as is generally required when performing ablation around more than one pulmonary vein ostium, as discussed above.

Tip 20 may be made from a transparent elastomer, and inflated (in a manner such as described above with regard to previous examples), for approximating tissue (and particularly pulmonary vein ostia of different diameters) while still allowing viewing through endoscope 16. When approximating a pulmonary vein ostium, tip 20 may be inflated to an outside diameter that prevents it from being inserted into the pulmonary vein and which insures that a lesion formed by ablation element 12 will not intersect the pulmonary vein ostium.

Optionally, an expandable support member 102, such as an inflatable balloon member or other expanding structure may be mounted at the distal end of tube 14 for providing support to ablation element 12 when in an expanded diameter configuration. A lumen or port (independent of the lumen or port used to inflate tip 20) is provided (such as through tube 14, for example) for applying fluid under pressure, from a source outside of instrument 10 and located proximally thereof or mounted on a proximal portion thereof, to expandable member 102 to inflate it. FIG. 15C shows expandable member 102 in the contracted position, where it closely profiles the outside diameter of tube 14 to facilitate insertion thereof through a small opening in a patient. When in balloon form, expandable member may be formed of an elastic polymer, such as those that may be used in forming tip 20, as discussed above, or of a relatively rigid polymer, such as those also described above, to provide additional support to ablation element 12. When the balloon is elastic, it may be constructed from silicone rubber, latex rubber, or polyurethane, for example. When the balloon is constructed from a relatively inelastic polymer, it may be constructed from polyethylene, PET (polyethylene terepthalate), a nylon-polyurethane composite, or the like.

Once distal tip 20 and the distal end portions of endoscope 16 and tube 14 (including expandable member 102) have been inserted beyond the small opening (such as an opening in an atrial appendage, for example), expandable member may be expanded (such as by inflating using pressurized saline when expandable member is a balloon member) as shown in FIG. 15D. Expandable member expands to an outside diameter at least as large as the outside diameter of ablation element 12 and typically to a outside diameter that is greater than that of ablation element 12, even when instrument 10 is in the fully telescoped in configuration, thereby provided support for ablation element as it is pressed against tissue to perform an ablation. The support provided by expandable member 102 helps keep ablation element 12 approximated to the tissue during performance of an ablation, especially in those configurations where the approximation requires contacting the ablation element to the tissue, reducing any tendency for ablation element 12 to deflect proximally back toward tube 14 under pressure.

Referring now to FIG. 16, a device 110 for facilitating the delivery of an instrument, such as an ablation instrument 10 through the atrial appendage 1 of a patient's heart is shown. Although device 110 is shown attached to the atrial appendage, it is noted that device 110 could be used in a similar manner to attach to an area of tissue through which an opening is desired to be formed, and for facilitating insertion of instruments through such an opening formed. Delivery guide 110 includes main tube 112 which it typically formed from a rigid plastic such as polycarbonate, liquid crystal plastic, ULTEM®, or the like, but may also be made from metal such as stainless steel or other biocompatible metal. Delivery guide 110 may be flexible or rigid, and typically has an outside diameter of about 10 to about 20 mm.

A sewing ring 114 is mounted to the distal end of tube 112. Sewing ring 114 may be made from a rigid plastic such as polycarbonate, liquid crystal plastic, ULTEM®, or the like, or from a flexible material such as fabric (made from nylon, TEFLON®, silk, and/or polyester), an elastomer such as silicone rubber or polyurethane, or a flexible plastic such as polyvinyl chloride, polyethylene or the like, or from combinations of any of the rigid, elastomeric or flexible polymers mentioned. Sewing ring 114 forms a border around tube 112, and extends about 5 to 10 mm in a circumferential fashion from the outer diameter of tube 112. Tube 112 is configured to have an inside diameter slightly larger than the largest instrument that is intended to be delivered through tube 110.

After forming an opening such as a thoracotomy in the patient, working down through the pericardium and locating the patient's atrium 9 and atrial appendage 1, device 110 is inserted through the opening to approximate the distal end of device 110 with the atrial appendage 1 and sewing ring 114 is sutured to the atrial appendage sufficiently to form a substantially leak proof seal between the atrial appendage 1 and tube 112. Device 110 further includes a hemostatic valve 116 mounted in a proximal end portion thereof, which seals the proximal end of device 110 thereby preventing any blood flow therethrough. When an instrument is inserted into tube 112 through valve 116, valve 116 also forms a hemostatic seal with the instrument, so that the combination of instruments also prevent blood flow through the proximal end of instrument 110 between the instruments.

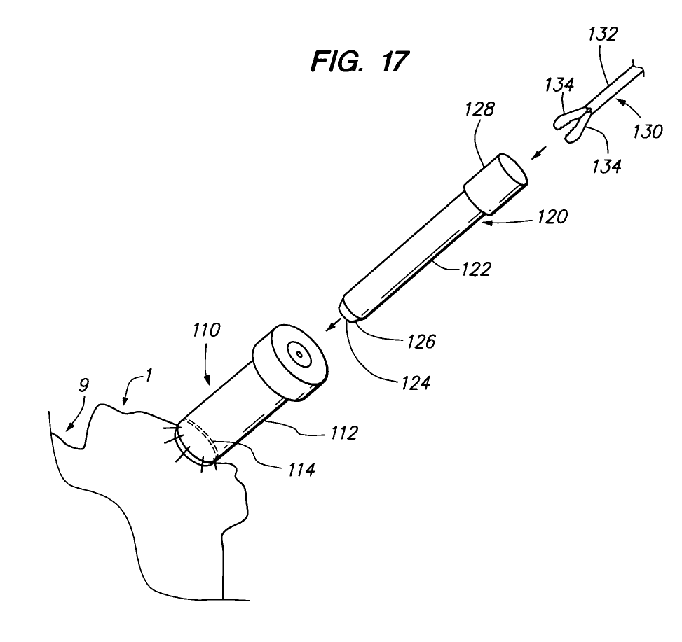

Referring now to FIG. 17, a tubular cutter 120 is provided for insertion through device 110 to cut an opening through the atrial appendage 1. Cutting device 120 may be made of a rigid tubular body 122, such as from rigid biocompatible plastic or biocompatible metal, and has an outside diameter only slightly smaller than the inside diameter of tube 112, so that tube 112 acts as a guide during rotation of cutter 120 during the performance of cutting the opening. The distal end of device 120 is provided with a sharp knife edge, and is typically formed from biocompatible metal. The distal end portion may be beveled 124 to facilitate cutting action. The proximal end portion 128 of device 120, may be formed to have an outside diameter at least slightly larger than the inside diameter of valve 116 in the fully opened position, to prevent inserting device 120 too far into device 110 as well as to facilitate manipulation if device 10 by the operator.

After insertion of cutter 120 into device 110 as described above, and prior to cutting an opening through the wall of atrial appendage 1, a thin-stemmed grasping instrument, such as grasper 130 is inserted through the tubular opening in cutter 120 to an extent to contact the tissue of the atrial appendage 1. The stem or shaft 132 of grasper 130 is of sufficient length so that the controls for operating the grasping jaws 134 (such as scissor handles or the like, not shown) extend out of the patient for easy manipulation by operator. Jaws 134 are of a size that permit them to be opened within the confines of the annulus of tube 122. Jaws 134 are contacted with the tissue of the atrial appendage, and then clamped shut to grasp the tissue. Next, the operator rotates cutter 120 until an opening has been cut through the atrial appendage. Once the opening has been fully cut, grasper 130 is withdrawn from cutter 120, while still grasping the severed tissue to remove it from the site. Cutter 102 is also withdrawn, leaving device 110 sutured to the atrial appendage, ready to receive other instruments for performing one or more surgical procedures.

At the completion of the procedure, the atrial appendage may be stapled and transected at the base of the appendage, using a stapling instrument such as an endoscopic GIA stapler (available from AutoSuture, United States Surgical Corporation, now part of Tyco Corporation, or from Ethicon Endosurgery, a Johnson and Johnson corporation). Alternatively, the base of the atrial appendage may be oversewn with sutures, and the sutures in the sewing ring may then be cut to allow removal of the device. Further alternatively the appendage may be oversewn with sutures and then the appendage may be amputated at its base, above the oversewn sutures. For example, ablation device 10 may be inserted to perform atrial ablation procedures as described above.

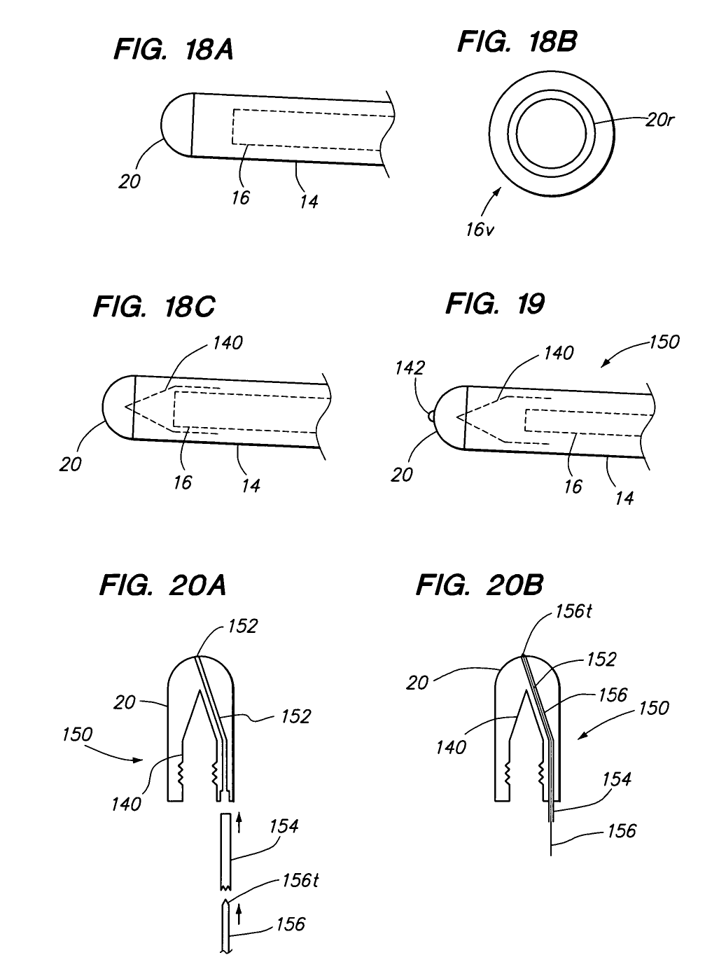

For devices employing an endoscope in a manner as described above, wherein the distal end of the endoscope 16 may be varied as to its distance from the distal tip 20 that it is viewing though, it has been observed that when the distal end of endoscope 16 is within the radius of tip 20 or near to tip 20 for narrower viewing fields, clear unobstructed views may be provided. However, in some instances, when the distal tip of endoscope 20 is retracted significantly from the radial confines of tip 20, as illustrated in FIG. 18A, to increase the focal length visualized, a bright ring 20r formed by a reflection off the spherical surface of tip 20 may appear in the view 16V provided through the proximal end of endoscope 16, see FIG. 18B. The provision of a tapered or conical tip 20 eliminates this artifact, but such a tip configuration may be generally unsuitable for endocardial applications as well as epicardial applications, as the risk of damaging tissue with a fairly acutely shaped tip may be too great.

FIG. 18C shows an instrument similar to that shown in FIG. 18A, except that a tapered or conical transparent tip 140 have been mounted concentrically within tube 14 and hemispherical tip 20 and around endoscope 16. The surface of angled or conical tip 140 breaks up the reflected waves from the blunt tip 20 and prevents the formation of the ring 20r in the visualization through endoscope 16. This configuration of a sharper tip 20 within a blunt tip 20 may be employed in ablation devices 10 that use a blunt tip 20 as described above, as well as other instruments designed to contact tissues while providing visualization.

One example of another such instrument is a dissection instrument 150, a distal portion of which is shown in FIG. 19. Dissection instrument 150 may be used, for example, to endoscopically dissect the pericardial reflection posterior to the superior vena cava and to access the transverse pericardial sinus for epicardial probe placement. Dissection instrument includes a rigid, transparent, blunt tip 20 that enables viewing of the progress of the dissection procedure through endoscope 16. A small, distal feature, such as a gauze (or alternatively, an extension of tip 20 made of the same material as tip 20) tip 142 having a diameter of about 1 mm and a length of about 2 mm may be provided at the distal end of tip 20 to aid in the dissection. For example, gauze provides friction against the tissue to facilitate blunt dissection. An inner conical tip 140 that has a fairly sharp or pointed distal end is provided concentrically within tip 20 to prevent the formation of a reflected ring 20r in the visualization by endoscope 16, while at the same time, blunt tip 20 facilitates blunt dissection of the tissues being dissected.

FIG. 20A shows a partial sectional illustration of another example of a dissection instrument 150 in which tube 14 and endoscope 16 are not shown for reasons of simplifying the illustration. In this example, an aspiration/irrigation channel 152 or lumen is provided to extend through tip portion 20 to a distal opening in tip 20. A tube or lumen 154 connects with aspiration/irrigation channel 152 and extends through instrument 150 to the proximal end portion thereof, for connection with a source of irrigation fluid, a suction source, or for other functions described below. Alternatively, an integral lumen, channel or tube may be used in place of channels/tubes 152 and 154. As noted with regard to FIG. 19, tip 20 is rigid, blunt and transparent, and may be formed of a rigid transparent plastic or glass, for example.

A stylet 156, which may have a sharpened distal tip 156t, having an outside diameter configured to allow stylet 156 to be freely slid within tube 154 and channel 152, and having a length sufficient to extend out of a proximal end portion of instrument 150 even when the distal tip extends from the distal end of tip 20, is insertable through tube 54 and channel 152. Such insertion may be carried out to clear channel 152 of clot formation and/or debris, which may accumulate during dissection. Additionally, when fully inserted, distal tip 156t may protrude slightly out of the distal face of dissection tip 20, as shown in FIG. 20B, to act as a small cleat for initiating a dissection.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of performing ablation transmurally across the wall of an organ, said method comprising the steps of:
preparing an opening in a patient to provide direct access to the wall of the organ;
preparing an opening through the organ;
inserting an ablation device through the opening in the patient and the opening through the organ;
approximating a target area of an inner wall of the organ with an ablation element of the ablation device; and
ablating the target area to create a lesion.

2. The method of claim 1, wherein the lesion is a transmural lesion.

3. The method of claim 1, wherein the organ is a heart.

4. The method of claim 3 wherein the organ is an atrium of the heart.

5. The method of claim 4, wherein the organ is a left atrium of the heart.

6. The method of claim 4, wherein said preparing an opening through the organ comprises creating an incision in the atrial appendage of the atrium.

7. The method of claim 3, wherein the opening is formed through the apex to gain access to the left ventricle.

8. The method of claim 7, wherein the target area is an inner wall of the left ventricle.

9. The method of claim 6, further comprising attaching a delivery guide to the atrial appendage to surround the incision and to provide a guide for insertion of the ablation device therethrough.

10. A method of performing atrial ablation, said method comprising the steps of:
making an opening in a patient to provide direct access to the heart of the patient;
making an opening in the pericardium;
inserting an ablation device through the opening in the patient and the opening in the pericardium;
approximating a target area of a wall of the organ with an ablation element of the ablation device; and
ablating the target area to create a lesion.

11. The method of claim 10, wherein said opening in a patient is a small thoracotomy.

12. The method of claim 10, wherein the heart continues to beat during the performance of all of said steps.

13. The method of claim 10, wherein the lesion is a transmural lesion.

14. The method of claim 10, wherein the target area approximated is on the epicardial wall of the atrium.

15. The method of claim 10, further comprising making an opening in the atrial appendage of the atrium, wherein said inserting an ablation device further comprises inserting the ablation device through the opening of the atrial appendage, and wherein the target area approximated is on the endocardial wall of the atrium.

16. The method of claim 15, wherein the target area includes endocardium around at least one pulmonary ostium.

17. The method of claim 15, further comprising attaching a delivery guide to the atrial appendage to surround the opening therein and to provide a guide for insertion of the ablation device therethrough.

18. The method of claim 17, further comprising applying a purse string suture around the atrial appendage, and tightening the purse string suture during removal of the delivery guide to reduce blood loss from the opening in the atrial appendage.

19. A method of performing atrial ablation, said method comprising the steps of:
inserting an ablation device comprising a rigid or malleable tube and at least one ablation element at a distal end portion thereof through an opening in the chest of a patient and through the atrium;
viewing the location and placement of the distal end of the ablation device through an endoscope passing axially therethrough; and
ablating tissue at a target location on the endocardium in the atrium.

20. The method of claim 19, further comprising monitoring said ablating, and ceasing said ablating when it is determined by said monitoring that a sufficient amount of ablation has been performed.

21. The method of claim 19, wherein the heart continues to beat during the performance of all of said steps.

22. The method of claim 20, wherein said monitoring comprises visual monitoring through the endoscope.

23. The method of claim 20, wherein said monitoring comprises contacting the tissue with at least one thermocouple in at least one location radially inward or outward of said at least one ablation element with respect to the ablation device, by a distance substantially equal to a thickness of a wall of the atrium in the target location.

24. The method of claim 19, wherein the at least one ablation element is placed radially beyond a periphery of a pulmonary ostium, and said ablating is performed to provide a ring-shaped lesion in the endocardium of the atrium surrounding the pulmonary ostium.

25. An ablation device for directly accessing a surgical site to perform ablation on a targeted tissue, said device comprising:
an elongated rigid or malleable tube having a distal end portion and a proximal end portion, said elongated tube having sufficient length so that at least a proximal end of the proximal end portion extends out of a patient when a distal end of the distal end portion contacts the targeted tissue;
an endoscope axially received within said elongated rigid or malleable tube;
a transparent tip closing the distal end of said distal end portion, wherein said transparent tip enables direct viewing through the distal end of the device using said endoscope; and
at least one ablation element mounted on said device at said distal end portion.

26. The ablation device of claim 25, wherein said at least one ablation element is mounted radially outside of a perimeter of said transparent tip.

27. The ablation device of claim 25, wherein said transparent tip is hemispherical.

28. The ablation device of claim 25, wherein said distal end portion has an outside diameter that is larger than an outside diameter of a portion of said tube adjacent said distal end portion, to permit said at least one ablation element to be mounted radially outside of the perimeter of said transparent tip on a distal end of said distal end portion.

29. The ablation device of claim 25, wherein said elongated tube is rigid.

30. The ablation device of claim 25, wherein said at least one ablation element comprises a circumferentially electrically conducting element mounted around a circumference of said distal end of said distal end portion.

31. The ablation device of claim 25, wherein said at least one ablation element comprises an arc-shaped electrically conducting element mounted on a portion of a circumference of said distal end of said distal end portion.

32. The ablation device of claim 25, wherein said at least one ablation element comprises a single point-shaped electrically conducting element mounted on a point location of a circumference of said distal end of said distal end portion.

33. The ablation device of claim 25, wherein said at least one ablation element is mounted inside of said distal end portion and is configured to conduct ablation energy to saline in contact therewith.

34. The ablation device of claim 25, wherein said transparent tip is sized to approximate an inside diameter of an ostium of a pulmonary vein located in a left atrium of a patient.

35. The ablation device of claim 25, wherein at least said distal end portion of said tube is articulatable with respect to said proximal end portion.

36. The ablation device of claim 25, further comprising a light emitter provided in said distal end portion configured to direct light out of the distal end of said device.

37. The ablation device of claim 25, further comprising a sliding ring configured to slide with respect to said tube, over said transparent tip such that at least a distal end of said sliding ring is positioned distally of said transparent tip, wherein said at least one ablation element is mounted on said distal end of said sliding ring.

38. The ablation device of claim 25, further comprising a sliding ring configured to slide with respect to said tube, said device configured to deliver saline into contact with said at least one ablation element, wherein said at least one ablation element is mounted inside of said sliding ring and is configured to conduct ablation energy to saline contacting said at least one ablation element.

39. The ablation device of claim 37, wherein said sliding ring is slidable to a retracted position where said transparent tip extends distally of said distal end of said sliding ring.

40. The ablation device of claim 25, wherein said endoscope is axially translatable with respect to said elongated tube to change a distance of a distal end of said endoscope from the distal end of said distal end portion of said device.

41. The ablation device of claim 25, wherein said transparent tip is formed of an elastomer.

42. The ablation device of claim 41, wherein said transparent tip is inflatable to about 300% to about 500% elongation of the elastomeric balloon material.

43. The ablation device of claim 37, wherein said sliding ring comprises a radially expandable ring, and said transparent tip is formed of an elastomer, said transparent tip being inflatable to expand a circumference thereof, wherein upon inflating said transparent tip to expand the circumference thereof, said expandable ring is also radially expanded.

44. The ablation device of claim 25, further comprising an additional tube coaxially positioned over said elongated rigid or malleable tube, wherein said at least one ablation element is mounted on a distal end portion of said additional tube.

45. The ablation device of claim 44, wherein said distal end portion of said additional tube comprises an expanding member.

46. The ablation device of claim 45, wherein, when in a contracted configuration, said expanding member is substantially tubular, and closely conforms to said additional tube, and when in an expanded configuration, said expanding member is substantially funnel-shaped, with the larger diameter portion of the funnel-shape at the distal end of said expanding member.

47. The ablation device of claim 46, wherein said transparent tip is inflatable, and wherein said expanding member positions said at least one ablation member radially outside of said transparent tip when said transparent tip is inflated and said expanding member is in said expanded configuration.

48. The ablation device of claim 45, wherein said expanding member comprises an expanding frame having eyelets through which said at least one ablation member is threaded.

49. The ablation device of claim 48, wherein said expanding frame has a sinusoidal configuration.

50. The ablation device of claim 45, further comprising a thin sheet of material covering said expanding frame to exclude fluids from passing through said expanding member and into a cavity defined therein.

51. The ablation device of claim 25, further comprising at least one thermocouple mounted at said distal end of said distal end portion in at least one location radially inward or outward of said at least one ablation element by a distance substantially equal to a thickness of a wall of an organ to be transmurally ablated by contact with the targeted tissue.

52. The ablation device of claim 25, wherein said transparent tip comprises a flexible, generally inelastic balloon that, when deflated may is gatherable about said tube to closely conform to a cross-section profile of said tube.

53. The ablation device of claim 52, wherein, when inflated, said transparent tip expands to an inflated configuration, said inflated configuration having an outside diameter substantially larger than an outside diameter of said tube.

54. The ablation device of claim 53, wherein said outside diameter of said transparent tip in said inflated configuration is larger than an inside diameter of a pulmonary vein ostium about which an ablation is to be performed.

55. The ablation device of claim 52, wherein said generally inelastic balloon comprises a generally inelastic polymer selected from the group consisting of: polyethylene, polyurethane, polyvinyl chloride and polyethylene terepthalate.

56. The ablation device of claim 52, wherein said at least one ablation element is mounted on a distal face of said generally inelastic balloon.

57. The ablation device of claim 56, wherein said at least one ablation element comprises a flexible ablation element.

58. The ablation device of claim 56, wherein said at least one ablation element is adhered to said distal face.

59. The ablation device of claim 53, wherein a distal end portion of said endoscope is positionable inside of said generally inelastic balloon in said inflated configuration such that an outline of said at least one ablation element is visible through said endoscope.

60. The ablation device of claim 25, wherein said at least one ablation element has an encircling configuration dimensioned to surround a pulmonary vein ostium without contacting or intersecting the pulmonary vein ostium.

61. The ablation device of claim 59, wherein said distal end portion of said endoscope is axially slidable with respect to said inelastic balloon.

62. The ablation device of claim 52, further comprising a protrusion extending from a distal face of said generally inelastic balloon.

63. The ablation device of claim 25, wherein said transparent tip is expandable to vary an outside diameter thereof.

64. The ablation device of claim 25, further comprising a mechanism for mechanically increasing or decreasing the outside diameter of said transparent tip.

65. The ablation device of claim 63, wherein said transparent tip comprises a conical lens, said conical lens being mounted to a coil, said coil being manipulatable to vary the outside diameter of said conical lens.

66. The ablation device of claim 65, wherein said coil comprises a first end mounted to said tube, and a second end mounted to a second tube provided coaxially within said tube and coaxially over said endoscope, wherein relative rotation between said tube and said second tube actuates said coil to vary the outside diameter of said conical lens.

67. The ablation device of claim 63, wherein said transparent tip comprises a lens having overlapping edges, wherein rotation of one of said edges with respect to another of said edges varies the outside diameter of said lens.

68. The ablation device of claim 63, further comprising a sealing sleeve extending between said transparent tip and said tube.

69. The ablation device of claim 66, further comprising a control mechanism for selectively maintaining said tube and said second tube in fixed positions relative to one another to maintain a desired outside diameter of said tip.

70. The ablation device of claim 25, wherein said transparent tip comprises an elastic tip member and said at least one ablation element comprises a single point element adapted to contact tissue and be circumscribed about an ablation site by rotation of said tube and said tip.

71. The ablation device of claim 70, wherein said elastic tip member is configured to be inflated to facilitate viewing therethrough and through said endoscope.

72. The ablation device of claim 25, wherein said transparent tip is rigid.

73. The ablation device of claim 72, wherein said at least one ablation element comprises an element adapted to be dragged over tissue to form a lesion pathway that follows the dragging of said element.

74. The ablation device of claim 72, wherein said at least one ablation element comprises a pair of electrodes mounted peripherally of said transparent tip.

75. The ablation device of claim 72, wherein said rigid transparent tip has a blunt shape.

77. The ablation device of claim 25, wherein said at least one ablation element comprises a variable diameter ablation element.

78. The ablation device of claim 77, wherein said variable diameter ablation element comprises a spiral member interconnected between said distal end of portion of said tube and said transparent tip, said tube being axially movable with respect to said tip to telescope said spiral member in and out to vary the outside diameter thereof.

79. The ablation device of claim 78, wherein said transparent tip comprises an elastic inflatable member.

80. The ablation device of claim 25, wherein said transparent tip comprised a blunt distal surface, said device further comprising a lens having a sharp configuration mounted between said transparent tip and a distal end of said endoscope.

81. The ablation device of claim 80, wherein said lens having a sharp configuration comprises a conical tip.

82. An ablation device for directly accessing a surgical site to perform ablation on a targeted tissue, said device comprising:
an elongated rigid or malleable tube having a distal end portion and a proximal end portion, said elongated tube having sufficient length so that at least a proximal end of the proximal end portion extends out of a patient when a distal end of the distal end portion contacts the targeted tissue;
an endoscope axially received within said elongated rigid or malleable tube;
a transparent tip axially aligned with said elongated tube, distally of said elongated tube, wherein said transparent tip enables direct viewing through the distal end of the device using said endoscope, and wherein said transparent tip is expandable to vary an outside diameter thereof; and
at least one ablation element mounted on said device for application of ablation energy to tissue when approximated by said device.

83. An ablation device for directly accessing a surgical site to perform ablation on a targeted tissue, said device comprising:
an elongated rigid or malleable tube having a distal end portion and a proximal end portion, said elongated tube having sufficient length so that at least a proximal end of the proximal end portion extends out of a patient when a distal end of the distal end portion contacts the targeted tissue; and
a variable diameter tip mounted to said distal end portion of said tube, said variable diameter tip adapted to contact tissue and apply at least one of an energy or chemical to the tissue to perform ablation of the tissue.

84. The ablation device of claim 83, further comprising an endoscope axially received within said elongated rigid or malleable tube;

85. The ablation device of claim 83, wherein said variable diameter tip comprises an expandable ring.

86. The ablation device of claim 85, wherein said expandable ring comprises an elastic spring coil.

87. The ablation device of claim 85, wherein said expandable ring functions as an ablation element.

88. The ablation device of claim 83, further comprising a plurality of pre-curved, elongated control members mounted to said variable diameter tip and extending into said tube, said elongated control members being slidable with respect to said tube to vary the diameter of said variable diameter tip.

89. The ablation device of claim 83, further comprising a plurality of secondary tubes passing within said elongated tube and configured to control movements of respective ones of said elongated control members therethrough.

90. The ablation device of claim 85, further comprising an expandable transparent diaphragm spanning said expandable ring.

91. The ablation device of claim 90, further comprising a sealing sleeve extending between said expandable ring and a distal end of said tube.

92. The ablation device of claim 88, further comprising a plurality of pre-curved, elongated control members mounted to said variable diameter tip and extending into said tube, and a sealing sleeve extending between said expandable ring and a distal end of said tube; said elongated control members being slidable with respect to said tube and said sealing sleeve to vary the diameter of said variable diameter tip.

93. The ablation device of claim 84, wherein said endoscope is axially slidable, relative to said tube to vary a location of a distal end of said endoscope among a range of locations between a distal end of said tube and said variable diameter tip.

94. The ablation device of claim 84, wherein a distal end portion of said endoscope is articulatable to provide panning of a view while viewing through said endoscope

95. The ablation device of claim 83, wherein said variable tip diameter comprises a lens having overlapping edges, wherein rotation of one of said edges with respect to another of said edges varies the outside diameter of said lens.

96. The ablation device of claim 83, wherein said elongated tube includes a distal end portion wherein said tubing is split.

97. The ablation device of claim 96, further comprising a second tube coaxially passing within said elongated tube; and elastic balloon member closing a distal end of said second tube; and an endoscope coaxially passing within said second tube.

98. The ablation device of claim 97, wherein said elastic balloon member comprises a substantially flat distal end.

99. The ablation device of claim 97, further comprising an expandable ring mounted over distal end portions of said split tubing.

100. An ablation device for directly accessing a surgical site to perform ablation on a targeted tissue, said device comprising:
an elongated rigid or malleable tube;
a transparent distal tip mounted at a distal end of said tube;
a balloon member axially mounted over a distal end portion of said tube, proximal of said distal tip and fluidly connected to an opening through said tube for inflation of said balloon member by delivering pressurized fluid through said tube; and
an ablation element located within said balloon member

101. The ablation device of claim 100, further comprising an endoscope passing coaxially through at least a portion of said tube, said ablation element being located concentrically outside of a distal end portion of said endoscope.

102. The ablation device of claim 100, wherein said ablation element is adapted to deliver ultrasonic energy through said pressurized fluid and said balloon member to perform the ablation.

103. A device for facilitating the formation of an opening through an organ and for facilitating the delivery of at least one instrument through the opening, said device comprising:
an elongated main tube having proximal and distal ends;
a sewing ring located about said distal end; and
a one-way valve located within a proximal end portion of said main tube.

104. The device of claim 103, wherein said elongated main tube has a length sufficient to extend from a surface of the organ and proximally out of a percutaneous opening formed in a patient.

105. The device of claim 103, wherein said one-way valve substantially prevents blood flow proximally therethrough.

100. A dissection instrument comprising:
an elongated rigid or malleable tube having a distal end portion and a proximal end portion, said elongated tube having sufficient length so that at least a proximal end of the proximal end portion extends out of a patient when a distal end of the distal end portion contacts tissue as it is dissected;
an endoscope axially received within said elongated rigid or malleable tube;
a transparent blunt tip closing the distal end of said distal end portion, wherein said transparent blunt tip enables direct viewing through the distal end of the device using said endoscope; and
a transparent member having a sharp configuration mounted between said blunt tip and a distal end of said endoscope.

107. The dissection instrument of claim 106, further comprising a protrusion extending distally from a distal end surface of said transparent blunt tip, said protrusion configured to facilitate dissection.

108. The dissection instrument of claim 106, wherein said transparent member having a sharp configuration comprises a conical lens.

109. The dissection instrument of claim 106, further comprising a channel extending through at least a portion of a length of said elongated tube and extending through said transparent blunt tip.

110. The dissection instrument of claim 109, wherein said channel extends through a distal surface of said transparent blunt tip.

111. The dissection instrument of claim 109, further comprising a stylet adapted to be passed through said channel to extend out of said blunt tip to facilitate dissection.

112. An ablation device for directly accessing a surgical site to perform ablation on a targeted tissue, said device comprising:
an elongated rigid or malleable tube having a distal end portion and a proximal end portion, said elongated tube having sufficient length so that at least a proximal end of the proximal end portion extends out of a patient when a distal end of the distal end portion contacts the targeted tissue;
an endoscope axially received within said elongated rigid or malleable tube;
a transparent tip closing the distal end of said distal end portion, wherein said transparent tip enables direct viewing through the distal end of the device using said endoscope; and
at least one ablation element mounted on a distal end portion of said device.

113. The ablation device of claim 112, wherein said transparent tip comprises a flexible, substantially inelastic balloon.

114. The ablation device of claim 113, wherein said balloon, when deflated, is gathered about said distal end portion to reduce a profile of said device.

115. The ablation device of claim 113, wherein, when inflated, said balloon has a diameter substantially greater than a pulmonary vein ostium of a patient, thereby preventing said balloon and any portion of said device proximal of said balloon from entering the ostium.

116. The ablation device of claim 113, wherein said at least one ablation element is a flexible ablation element mounted on a distal surface of said balloon.

117. The ablation device of claim 113, wherein when said balloon is inflated, a distal end of said endoscope is positioned within said balloon.

118. The ablation device of claim 117, wherein when said distal end of said endoscope is axially positionable with said balloon to change a visual field viewed through said endoscope.

119. The ablation device of claim 113, wherein when said balloon comprises a protruding nipple on a distal face thereof.
